(19) United States
(12) Patent Application Publication
(10) Pub. No.: US 2026/0325563 A1
(43) Pub. Date: Oct. 22, 2026

(54) COMPOSITIONS AND METHODS FOR PROMOTING IN VIVO WOUND HEALING AND TISSUE REGENERATION

(71) Applicant: GODFRED Lubano Masinde, Brentwood, CA (US)

(72) Inventor: GODFRED Lubano Masinde, Brentwood, CA (US)

(21) Appl. No.: 19/174,725

(22) Filed: Apr. 9, 2025

(51) Int. Cl. A61K 31/7105; A61K 38/18; A61K 38/19; A61P 17/02

(52) U.S. Cl. CPC A61K 31/7105; A61K 38/18; A61K 38/19; A61P 17/02

(57) ABSTRACT

The presently disclosed subject matter provides compositions and methods for promoting in vivo cell proliferation, with applications in rapid wound healing and tissue regeneration. These methods involve the use of a cell proliferation composition containing small interfering RNA (siRNA) designed to target the 3′ untranslated region (UTR) of the aryl hydrocarbon nuclear transporter 2 (ARNT2) gene. By regulating ARNT2 expression, the disclosed compositions modulate key pathways involved in tissue repair, including inflammation, cell migration, and extracellular matrix remodeling, thereby enhancing the healing process and supporting tissue regeneration.

### CLAIM OF PRIORITY

This application claims the benefit of U.S. Provisional Patent Application Ser. No. 63/715,606, filed on Nov. 3, 2024. The entire disclosure of the referenced provisional application is hereby incorporated by reference in its entirety.

### GOVERNMENT INTERESTS

### Federally Sponsored Research or Development

The present invention was not made with any government funding or support.

### FIELD OF INVENTION

The present invention relates to the field of biomedical therapeutics and genetic medicine, and more particularly to the use of siRNA derived from the 3′ untranslated region (3′ UTR) of ARNT2 for promoting wound healing and facilitating tissue regeneration.

### BACKGROUND

Wound healing in mammals is a complex and prolonged repair process which often results in formation of fibrotic scars. Such scars can in turn impair tissue function. For example, healing processes in humans, particularly those involving trauma, acute illness, or major surgery, often result in chronic wounds or fibrotic scars that significantly affect tissue functionality. In contrast, amphibians are known for their ability to heal wounds rapidly without scarring. Such wound healing ability is attributable to the coordinated actions of various tissues and cell types. The process requires precise orchestration of cell migration, proliferation, matrix deposition, remodeling, inflammation, and angiogenesis.

Understanding the molecular pathways involved in promoting rapid and scarless healing is important for the development of therapeutic interventions effective in enhancing wound healing and preventing scarring. A variety of technologies have been developed to address this challenge. One approach involves modulating gene expression, such as by the application of siRNA, or portions thereof, to a subject, to promote faster and more efficient tissue regeneration.

### SUMMARY

The presently disclosed subject matter includes compositions and methods for promoting in vivo cell proliferation. The subject matter can be applied, for example, in rapid wound healing and tissue regeneration. Aspects of the methods include applying, in a cell proliferation composition, small interfering RNA (siRNA), such as a synthetic siRNA, that targets the aryl hydrocarbon nuclear transporter 2 (ARNT2) gene 3′ UTR, or a portion thereof. Resulting regulation of ARNT2 expression modulates pathways of tissue repair, including inflammation, cell migration, and extracellular matrix remodeling.

In various embodiments, the siRNAs are fully or partially double stranded. In some versions, the siRNA has a length of 50 or fewer nucleotides, e.g., 40 or fewer nucleotides, e.g., 30 or fewer nucleotides, e.g., 25 or fewer nucleotides. In some aspects, the siRNA has a sequence selected, for example, from SEQ ID NO:1, SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, and SEQ ID NO:14, or has a sequence at least 50%, at least 75%, at least 80%, at least 75%, at least 80%, or at least 90%, identical to a sequence selected from SEQ ID NO:16, or SEQ ID NO:17. In some aspects, the siRNAs of the subject embodiments decrease expression levels of an ARNT2 when bound to the ARNT2, 3′UTR. Accordingly, the methods include decreasing such expression levels strategically.

In some versions, a pharmaceutically acceptable carrier is included in the subject compositions, e.g., a topical administration carrier and/or an injectable carrier. The subject compositions can be or include a topical gel, cream, hydrogel, or self-delivering siRNA system. The compositions can also include liposomes encapsulating the siRNA and/or the pharmaceutically acceptable carrier. The compositions can, in various aspects, include pharmaceutical delivery nanoparticles.

According to some aspects, the compositions have a 3′ UTR with a length of 300 bp or less. In various embodiments, the compositions include liposomes, nanoparticles, gels, and/or hydrogels. The compositions may also include RNA which is a self-delivery siRNA. In some versions, the siRNA has a concentration in the composition of 100 ng to 300 ng. Compositions according to the subject embodiments can also include growth factors, anti-inflammatory agents, and/or matrix metalloproteinase inhibitors. In some aspects, the 3′ UTR comprises a single nucleotide polymorphism (SNP), or a plurality thereof.

Methods are also included in the subject disclosure. Such methods include methods for promoting in vivo cell proliferation, cell generation, and/or cell regeneration. Such methods may also be applied to tissues. They may include administering an amount, e.g., a therapeutically effective amount, of small interfering RNA (siRNA) targeting a 3′ untranslated region (UTR) of ARNT2 gene of a subject and thereby decreasing expression of ARNT2 in the subject.

In various instances, the administering is performed topically to a wound site of the subject. Or the administering may be performed by injecting the siRNA into the subject. The administering may also be made via one or more delivery vehicle selected from nanoparticles, liposomes, hydrogels, a self-delivering siRNA, or another pharmaceutical carrier, or any combination thereof.

The administering can, in various embodiments, be performed to a wound site of the subject, and wherein the wound site is a surgical, traumatic, chronic, or burn wound site. The methods may include treating such a wound using the subject compositions. The administering may also include applying growth factors, anti-inflammatory agents, or matrix metalloproteinase inhibitors to the subject. The methods also, in various instances, include decreasing inflammation, increasing cell migration, and/or improving extracellular matrix remodeling in the subject.

Methods of manufacturing the subject compositions are also included herein. Such methods may include generating the subject compositions and packaging the cell proliferation compositions.

Additionally, the subject embodiments include methods for identifying a candidate compound for treatment of a wound site of a subject. The methods can include contacting a sample comprising an ARNT2 gene with a test compound comprising a small interfering RNA (siRNA) targeting a 3′ untranslated region (UTR) of the ARNT2 gene; determining if the test compound decreases ARNT2 expression level in the sample; and/or selecting the test compound as a candidate if the ARNT2 expression level is decreased.

Kits are also included herein. Such kits include: a composition including a small interfering RNA (siRNA) targeting a 3′ untranslated region (UTR) of an ARNT2 gene of a subject comprising one or more single nucleotide polymorphisms (SNPs); a buffer solution; and/or packaging.

These and other objects, advantages, and features of the disclosed subject matter will become apparent to those persons skilled in the art upon reading the details of the devices and methods as more fully described below.

### BRIEF DESCRIPTION OF THE FIGURES

The disclosed subject matter may be best understood from the following detailed description when read in conjunction with accompanying drawings. Included in the drawings are the following figures:

FIG. 1 provides a diagram illustrating a quantitative trait locus (QTL) on chromosome 7, identified through a whole genome scan. The QTL harbors the ARNT2 gene, a candidate involved in promoting wound healing and tissue regeneration.

FIG. 2 is a bar graph providing the results from crossing a parent good healer (MRL) with an F1 cross. This cross resulted in F2 mice exhibiting either extremely fast and scarless wound healing or extremely poor wound healing, worse than the poor healer parent (SJL). The mRNA from the extreme good healers and the extreme poor healers was extracted, and specifically using PCR products from the 3′ UTR of the ARNT2 gene, were sequenced. The sequences were compared between the extreme good healers (Panel A) and the extreme poor healers (Panel B).

FIG. 3 provides a diagram comparing the sequences of the 3′ UTR from good healers (Panel A) and poor healers (Panel B). This figure highlights the sequencing data for the 3′ UTR of the ARNT2 gene, showcasing specific mutations that segregate with either good or poor wound healing outcomes.

FIG. 4 provides a schematic of a structure of ARNT2 mRNA embodiment, highlighting the 3′ untranslated region (UTR) that contains identified single nucleotide polymorphisms (SNPs). This figure also marks the region covered by PCR primers used for sequencing, providing insight into the specific areas targeted for genetic analysis to reveal variations associated with wound healing efficacy.

FIG. 5 provides a line graph illustrating the rate of ear wound healing in the parent models, MRL (good healer) and SJL (poor healer), compared to their F1 offspring, which were a cross between the two. The healing process was monitored from 24 hours to 28 days. The F1 offspring exhibited a wound healing rate that was intermediate between the good healer (MRL) and the poor healer (SJL), suggesting a combination of genetic traits from both parent models.

FIG. 6 is a graph illustrating rates of wound healing in good healers and poor healers, compared to good healers treated with ARNT2 siRNA targeting the 3′ UTR region. The good healers (MRL) and poor healers (SJL) were ear-punched, and ARNT2 siRNA was topically applied to the wounds of one set of MRL, with the other MRL and SJL groups serving as controls. The healing process was monitored from 24 hours to 21 days. The MRL mice with siRNA from 3′UTR applied healed faster than MRL or SJL with gel control.

FIG. 7 provides a graph depicting the rate of wound healing in the good healer with siRNA added in the wound on the right side of the back compared to the left side of the same model, ARNT2 siRNA was topically applied to the wounds and the healing process was monitored from 6 hours to 8 days. The wound that siRNA was applied healed much faster than the one with control gel, indicating downregulation of ARNT2 promotes wound healing.

### DETAILED DESCRIPTION

The presently disclosed subject matter includes compositions and methods for promoting in vivo cell proliferation. The subject matter can be applied, for example, in rapid wound healing and tissue regeneration. Aspects of the methods include applying, in a cell proliferation composition, small interfering RNA (siRNA) that targets the aryl hydrocarbon nuclear transporter 2 (ARNT2) gene. Resulting regulation of ARNT2 expression modulates pathways of tissue repair, including inflammation, cell migration, and extracellular matrix remodeling.

Before embodiments of the disclosed subject matter are described in greater detail, it is to be understood that this disclosed subject matter is not limited to particular embodiments described, and as such may, of course, vary. It is also to be understood that the terminology used herein is for the purpose of describing particular embodiments only, and is not intended to be limiting, since the scope of the embodiments of the invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the embodiments of the invention. The upper and lower limits of these smaller ranges may independently be included in the smaller ranges and are also encompassed within the embodiments of the invention, subject to any specifically excluded limit in the stated range. Where the stated range includes one or both limits, ranges excluding either or both of those included limits are also included in the embodiments of the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the embodiments of the invention belong. Although any methods and materials similar or equivalent to those described herein can also be used in the practice or testing of the present embodiments of the invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the presently disclosed subject matter is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the disclosed subject matter and their specific spatial characteristics and/or abilities include those shown or substantially shown in the drawings or which are reasonably inferable from the drawings.

As will be apparent to those of skill in the art upon reading this disclosure, each of the individual embodiments described and illustrated herein has discrete components and features which may be readily separated from or combined with the features of any of the other several embodiments without departing from the scope or spirit of the presently disclosed subject matter. Any recited method can be carried out in the order of events recited or in any other order which is logically possible.

In further describing the instant subject matter, embodiments of compositions are described first in greater detail, followed by a description of methods that find use in employing the subject compositions in accordance with the embodiments of the disclosed subject matter, as well as a review of certain applications in which the embodiments of the disclosed subject matter find use.

### Compositions

Embodiments of the subject disclosure include compositions, such as pharmaceutical compositions, such as cell proliferation compositions, such as in vivo cell proliferation compositions. Such compositions can be gene modulation, e.g., gene downregulation compositions. Such compositions can encourage cell generation and/or re-generation when applied to a subject. As such, the compositions are wound healing such that they may cause a wound to heal faster than the wound would in the absence of such a composition. The compositions can also be applied to promote tissue regeneration where a normal healthy tissue has suffered a trauma, such as a wound, and is undergoing a process of regeneration toward its natural healthy state. The tissue can be dermal and/or muscle tissue and/or another tissue. The subject compositions also can reduce scar tissue formation in the healing process by reducing the amount of scar tissue formed compared to the amount formed in the absence of the composition.

In various embodiments, the subject compositions include an RNA, e.g., a small interfering RNA (siRNA), such as a synthetic siRNA. In some aspects the siRNA is configured to target a 3′ untranslated region (UTR) of an aryl hydrocarbon nuclear receptor translocater 2, “ARNT2” gene of a subject including one or more single nucleotide polymorphisms (SNPs), and/or a pharmaceutically acceptable carrier. In the targeting process, the siRNA may bind to the gene and modulate function of the gene, such as by downregulating its expression. Accordingly, in some versions, activity of ARNT2 in a solution including the siRNA may be less than what its activity would be in the absence of the siRNA.

In some versions of the subject embodiments, the small interfering RNA is a short interfering RNA. In some aspects, the siRNA is a double stranded molecule in solution, e.g., fully double stranded or partially double stranded and/or partially single stranded. In various embodiments, the subject siRNAs decrease expression and/or activity levels of an ARNT2 when bound to the ARNT2, e.g., bound to the ARNT2 in vivo.

As set forth herein, any vector or portion thereof, can be synthetic and/or non-naturally occurring, such as a synthetic nucleic acid, such as synthetic DNA, or synthetic RNA, such as synthetic siRNA. Synthetic refers to the process of being made using a laboratory or scientific generating procedure rather than appearing in nature. Synthesis of a nucleic acid may include binding one nucleotide, or a plurality of nucleotides, at a time to one another and/or to an existing chain to generate a synthetic sequence. Synthesis of a nucleic acid includes putting nucleotides together in a container and/or a solution and providing conditions, such as temperature and/or pressure conditions so that they bind to one another to form chains. In various embodiments, a synthetic nucleic acid is generated by binding two sequences, sense and antisense nucleic acids to one another. In some variations, a subject siRNA can be entirely synthetic.

The presently disclosed compositions include siRNA that target the 3′ UTR of the ARNT2 mRNA transcript. In some versions, 3′ UTR sequences are used in designing the subject siRNAs. The compositions can include siRNAs designed across the 3′ UTR that may be applied singly or in a combination with other individual designed siRNA sequences. For example, a composition may include siRNAs only having a sequence covering one part of the 3′ UTR or a combination of siRNAs spanning a whole 3′UTR, or any portions thereof, or any combination thereof.

An embodiment of a ARNT2 3′ UTR sequence used to design the subject siRNAs is set forth below as SEQ ID NO: 17. The siRNA embodiments target the 3′ UTR provided or any portion thereof. The target region, e.g., a region of binding complementarity, can have a length of, for example, any of the siRNAs described below. As provided herein, design and use of siRNAs based on this sequence includes decreasing ARNT2 expression in vitro which in turn leads to scarless wound healing, and provides the in vivo data as set forth herein.

| Homo sapiens ARNT2 3′UTR Target Sequence (SEQ ID NO: 17): | |
|---|---|
| 3985 | CTGCGGCCAGAGTGAGGCTCCTGCTGTACAGTGCC |
| 4021 | ACCCATACTGTGATGTCGATGCCCATGTGAATGAGGCCCACCCTCGCCCTGCTTGCCCTG |
| 4081 | CCGCAGGCCCCCCACCAGAAGCCATCTCCCCCGCTGTGTGTCCCCAGGCGCATCATTGCT |
| 4141 | CCACTCTCCCCTGCAGCCGCGGCTGCTCGGCCACTGACCAGGGGCCCTGGCAGACATTAG |
| 4201 | GGGATCAGTTGTATTTATTGTATTTTATCTGTGTGTGCTC AGGAGGTGAG CCTAGTGGGT |
| 4261 | CCTTAAGTTT GGTTGTGAAT AAT |

The nucleotide sequence comprises a portion of the 3′ untranslated region (UTR) of the Homo sapiens ARNT2 gene. This sequence is characterized by its ability to serve as a target site for sequence-specific RNA interference (RNAi) molecules. The sequence is identified as SEQ ID NO: 17 and spans the designated region within the ARNT2 3′ UTR.

In some versions of the subject embodiments, a 3′ UTR, such as that shown in by SEQ ID NO: 17, or provided herein by SEQ ID NO: 15 and SEQ ID NO: 16 includes one or a plurality of single nucleotide polymorphisms, “SNPs”. The embodiments may include, for example, 20 or fewer SNPs, 15 or fewer SNPs, 10 or fewer SNPs, 5 or fewer SNPs, 4 or fewer SNPs, 3 or fewer SNPs, 2 or fewer SNPs, or 1 SNP. The embodiments may include, for example, 20 or more SNPs, 15 or more SNPs, 10 or more SNPs, 5 or more SNPs, 4 or more SNPs, 3 or more SNPs, 2 or more SNPs, or 1 SNP or more SNPs. The embodiments may include a number of SNPs ranging from, for example, 1 to 15, such as 1 to 10, such as 1 to 5, or 1 to 3. In various embodiments, the subject 3′ UTR sequences may have a sequence identity 99% or greater, 98% or greater, 95% or greater, or 90% or greater identical to the sequence of SEQ ID NO: 17, the sequence of SEQ ID NO: 15, or the sequence of SEQ ID NO:16.

The presently disclosed compositions include specific siRNA sequences in Table 1, e.g., SEQ ID NOs: 1-14, that target the 3′ UTR of the ARNT2 mRNA transcript. The compositions can include siRNAs having any one or combination of the sequences set forth as SEQ ID NOs: 1-14. For example, a composition may include siRNAs having the sequence of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, or any combination thereof.

In various embodiments, the siRNAs are fully or partially double stranded. In some versions, the siRNA has a length of 50 or fewer nucleotides, e.g., 40 or fewer nucleotides, e.g., 30 or fewer nucleotides, e.g., 25 or fewer nucleotides. In some aspects, the siRNA has a sequence at least 50%, at least 75%, at least 80%, at least 75%, at least 80%, a least 85%, at least 90%, or at least 95% identical to a sequence selected from any of the sequences set forth in Table 1. In some aspects, siRNAs have sequences of two or three of the sequences set forth in Table 1. For example, siRNAs may include a combination of adjoined sequences of: SEQ ID NO: 1 and SEQ ID NO:2, SEQ ID NO:3 and SEQ ID NO:4, SEQ ID NO:5 and SEQ ID NO:6, SEQ ID NO:7 and SEQ ID NO:8, SEQ ID NO:9 and SEQ ID NO:10, SEQ ID NO:11 and SEQ ID NO: 12, SEQ ID NO: 13 and SEQ ID NO:14, or any portions of any of such sequence of a provided sequence pair. Embodiments of siRNAs may also include a combination of adjoined sequences of: SEQ ID NO:2 and SEQ ID NO: 3, SEQ ID NO:4 and SEQ ID NO:5, SEQ ID NO:6 and SEQ ID NO:7, SEQ ID NO: 8 and SEQ ID NO:9, SEQ ID NO:10 and SEQ ID NO:11, SEQ ID NO:12, SEQ ID NO: 13 and SEQ ID NO:14, or any portions of any of such sequence of a provided sequence pair.

The subject embodiments of siRNAs may be a combination of a first sequence in Table 1, or a portion thereof, e.g., a portion having a length of 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 nucleotides, and a second sequence in Table 1, or a portion thereof wherein the second sequence or portion thereof is adjoined to and continuous with the first and has a length of 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 nucleotides.

Table 1 provides embodiments of siRNA sequences targeting the 3′ UTR. As provided herein, application of siRNA having such sequences includes decreasing ARNT2 expression in vitro which in turn leads to scarless wound healing, and confirms the in vivo data as set forth herein. siRNA sequence numbers are provided in the Table 1 as SEQ ID 1-14.

TABLE 1
siRNA Targeting ARNT2, 3′ UTR
Sequence (SEQ ID 1-14)

| SiRNA No. | Sequence | SEQ ID NO |
|---|---|---|
| 1 | GUACUUUAUCUGUGUGCUC | 1 |
| 2 | CCAUGUUCAAAGAGGGUCC | 2 |
| 3 | AGCUUGCCCUGCUGUAGAA | 3 |
| 4 | CCCCAUCCUCCCUGGACAU | 4 |
| 5 | GUUAAGAAGUUGAGGGUAA | 5 |
| 6 | GAACUAACUUCUAGAACAA | 6 |
| 7 | GGGCAGAGCAAGCAGGAAA | 7 |
| 8 | GAAUGAGCCUGGAGAUUAA | 8 |
| 9 | GCUUUAAACCUGAGAAUCA | 9 |
| 10 | GAGCAGAGGUGGAGGAUUG | 10 |
| 11 | GGACAGCUUUGAAGACAUA | 11 |
| 12 | ACCUUUAGCUUGUGAAUUA | 12 |
| 13 | GGGAGAAGUCAGAAAUGAA | 13 |
| 14 | GGGUAGUAGUAUUUGGAUU | 14 |

In some embodiments the subject siRNAs have a length of, for example, 40 or fewer, 30 or fewer, 25 or fewer, 20 or fewer, 15 or fewer, 10 or fewer, or 5 or fewer, nucleotides. In some embodiments the subject siRNAs have a length of, for example, 40 or more, 30 or more, 25 or more, 20 or more, 15 or more, 10 or more, or 5 or more, nucleotides. A length of as subject siRNA may be any of: 1, 2, 3, 4, 5, 6, 7, 8, 9, 10, 11, 12, 13, 14, 15, 16, 17, 18, 19, 20, 21, 22, 25, 24, 25, 26, 27, 28, 29, or 30 nucleotides. Aspects of the subject siRNAs may have a length ranging, for example, from 10 to 30 nucleotides, such as 15 to 25 nucleotides, such as 19 to 25 nucleotides.

When ARNT2 and a subject siRNA are bound together in a complex, such as by placing them together in solution and/or in vivo, they may have a region of complementarity having a length of, for example, 40 or fewer, 30 or fewer, 25 or fewer, 20 or fewer, 15 or fewer, 10 or fewer, or 5 or fewer, nucleotides. In some embodiments, such a complementarity region has a length of, for example, 40 or more, 30 or more, 25 or more, 20 or more, 15 or more, 10 or more, or 5 or more, nucleotides. A length of such a complementary region may be any of: 1, 2, 3, 4, 5, 6, 7, 8, 9, 10, 11, 12, 13, 14, 15, 16, 17, 18, 19, 20, 21, 22, 25, 24, 25, 26, 27, 28, 29, or 30 nucleotides. In some embodiments, such a complementarity region has a length ranging, for example, from 10 to 30 nucleotides, such as 15 to 25 nucleotides, such as 19 to 25 nucleotides.

### Sequence Listing Incorporation by Reference

The Sequence Listing XML file submitted with this application is hereby incorporated by reference in accordance with 37 CFR 1.835 (a) (2) or 1.835 (b) (2). The details of the XML file are as follows:
File Name: ARNT2-ST.26 sequence-listing Apr. 4, 2025
Date of Creation: Mar. 20, 2025
File Size: 40,277 bytes

ARNT2 is part of the basic helix-loop-helix/PAS (bHLH/PAS) family of proteins, which are key regulators of transcriptional responses during development and in reaction to environmental stimuli. These proteins can function as heterodimers, and various dimeric combinations form in vivo to regulate gene expression under hypoxic conditions. For example, ARNT2 forms transcriptionally active heterodimers with hypoxia-inducible factor 1-alpha (HIF-1 alpha) and aryl hydrocarbon receptor (AHR), which are involved in regulating cellular responses to hypoxia. Mutations in the murine ARNT2 locus result in embryonic lethality by day 10.5 due to defects in placental vascularization and hematopoiesis.

Embodiments of the subject compositions can be in a variety of forms including a topical gel, cream, hydrogel, and/or self-delivering siRNA system. Accordingly, the compositions can be a liquid or a solid when administered to a subject, e.g., a patient. In some versions, the compositions include liposomes, such as microvesicles, which may encapsulate, for example, the siRNA and/or a pharmaceutically acceptable carrier. In some version, including liposomes in the composition enhances stability of the compositions and/or promotes cellular uptake. In some versions, the compositions include nanoparticles, e.g., pharmaceutical delivery nanoparticles, and/or hydrogels.

The subject compounds, e.g., siRNA-based therapies herein, may be combined with or include other therapeutic agents that enhance wound healing, such as growth factors, anti-inflammatory agents, and/or matrix metalloproteinase inhibitors. The combination of these agents with ARNT2-targeting siRNA from 3′ UTR can further accelerate the healing process and improve the quality of tissue regeneration.

In some embodiments, the siRNA is configured to target 3′ untranslated region (UTR) of an ARNT2 gene of a subject comprising one or more siRNAs across the gene. In various aspects, siRNA could be designed to cover any part of the 3″ UTR and used either as single or in combined.

In some versions of the subject embodiments, the UTR, e.g., the 3′ UTR, has a length of 350 bp or less, such as 300 bp or less, such as 275 bp or less. In various embodiments, the UTR has a length of 200 bp or greater, such as 250 bp or greater, such as 270 bp or greater, such as 275 bp or greater. In some versions, the UTR, e.g., the 3′UTR, has a length ranging from 200 bp to 350 bp, such as 250 bp to 300 bp, such as 270 bp to 280 bp.

Aspects of the embodiments include those wherein a pharmaceutically acceptable carrier is a topical administration carrier and/or an injectable carrier. In some versions, the siRNA is a self-delivering siRNA and/or includes Accell siRNA. Accell siRNA is a self-delivering siRNA that is specially modified for use without transfection reagent and works at a higher concentration than conventional siRNA with minimal disruption of the expression profile (Hedge et al, 2014).

In some embodiments, the siRNA has concentration, such as a concentration in the composition ranging from 50 ng to 400 ng, 100 ng to 300 ng, and/or 80 ng to 320 ng. Embodiments of the compositions include those wherein the siRNA has a concentration, such as a concentration in the composition, of 400 ng or less, 300 ng or less, or 200 ng or less. Embodiments of the compositions also include those wherein the siRNA has a concentration, such as a concentration in the composition, of 100 ng or greater, 150 ng or greater, or 200 ng or greater.

In some embodiments, the subject siRNAs are at least 50%, such as at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, spanning across the 3′ UTR ARNT2 sequence.

As set forth herein, a “subject” can be a “mammal” or “mammalian,” where these terms are used broadly to describe organisms which are within the class mammalian, or the subject can be non-mammalian, which are organisms not within the class. In some embodiments, subjects are humans. While the methods described herein can be applied to perform one or more protocol on a human subject or tissue thereof, it is to be understood that the subject methods can also be carried-out to perform a procedure and/or treatment on other subjects, that is, in “non-human subjects”, or a tissue thereof. Such subjects may include non-mammalian animals, bacteria, viruses, insects or plants. As such, cells according to the embodiments may be cells of tissues of one or more non-mammalian animals, bacteria, viruses, insects or plants. Any of the cells and/or cell populations set forth herein can be eukaryotic, prokaryotic, reproductive, non-reproductive, haploid, non-haploid and/or diploid cells.

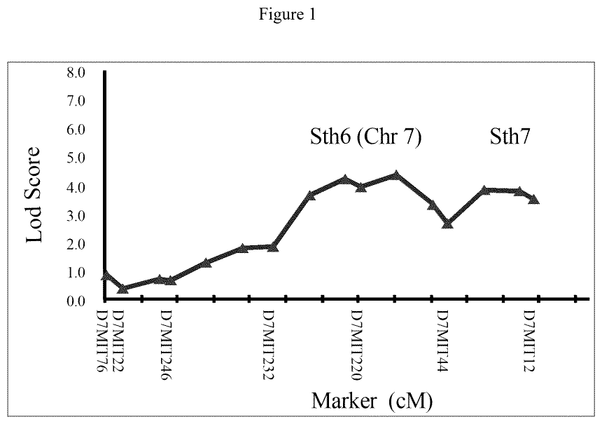

According to various embodiments of the disclosed subject matter, FIG. 1 provides a diagram illustrating the discovery of a quantitative trait locus (QTL) on chromosome 7, identified through a whole genome scan using interval mapping analysis several QTLs including the two QTLs on Chromosome 7 (Sth6 and Sth7) (Masinde et al, 2001). The QTLs identified on chromosome 7 in MRL/SJL F2 cross were identified in MRL/B6 F2 population (McBrearty et al. 1998). Accordingly, chromosome 7 was the focus of the subject studies for analysis of candidate genes for scarless wound healing. Over 15 genes were examined from chromosome 7 for SNPs using Roche SNP database, comparing different strains of mice, the 3′ UTR of the ARNT2 gene in the mice was the only gene found to have SNPs. There are two QTLs on chromosome 7, the first QTL, Sth6 (soft tissue healing) with peak marker D7Mit220 (38.3 cM) and between D7Mit232 (26.2 cM) and D7Mit 44 (50.3 cM) harbors ARNT2 (30.6 cM), a candidate gene involved in wound healing and tissue regeneration.

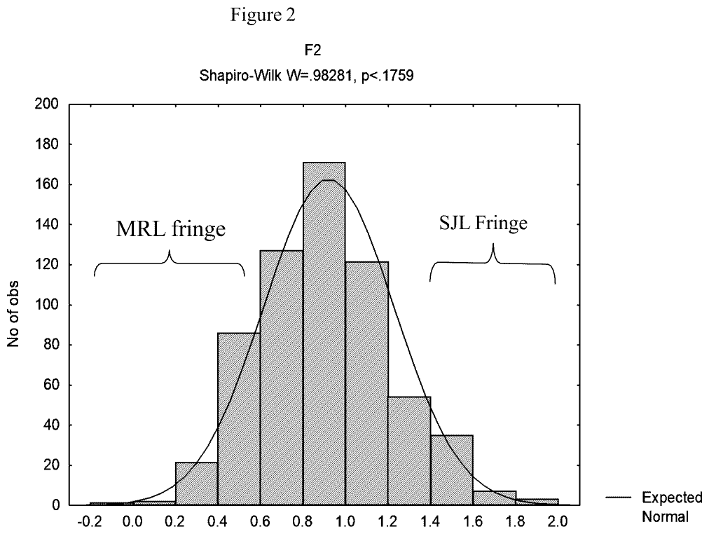

FIG. 2 is a bar graph illustrating the results from crossing the parent good healer (MRL) with the F1 cross. This cross resulted in F2 mice exhibiting either extremely fast and scarless wound healing or extremely poor wound healing, worse than the poor healer parent (SJL). The mRNA from the extreme good healers and the extreme poor healers was extracted, and specifically using PCR products from the 3′ UTR of the ARNT2 gene, were sequenced. The sequences were compared between the extreme good healers (Panel A) and the extreme poor healers (Panel B). The sequencing highlights show that the 3′ UTR of the ARNT2 gene contains specific mutations that segregate with either good or poor wound healing outcomes. These mutations are mapped and distinguished across the eighteen sequences, emphasizing their correlation with the wound healing abilities of the subjects.

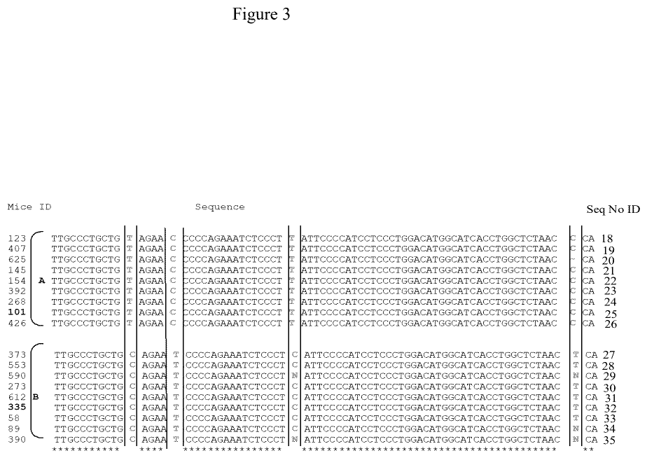

FIG. 3 provides a diagram comparing the sequences of the 3′ UTR from good healers (Panel A) and poor healers (Panel B). This figure highlights the sequencing data for the 3′ UTR of the ARNT2 gene, showcasing specific mutations that segregate with either good or poor wound healing outcomes. The mutations are mapped and distinguished, in the eighteen sequences, emphasizing their correlation with the wound healing abilities of the subjects.

According to various embodiments, FIG. 3 shows eighteen aligned sequences for ARTN2 from fast wound healers and poor healers from MRL/SJL F2 cross, highlighting the 3′ untranslated region (UTR) that contains identified single nucleotide polymorphisms (SNPs) that are specific for good healers (panel A) or poor healers (panel B) (SEQ ID 18-35).

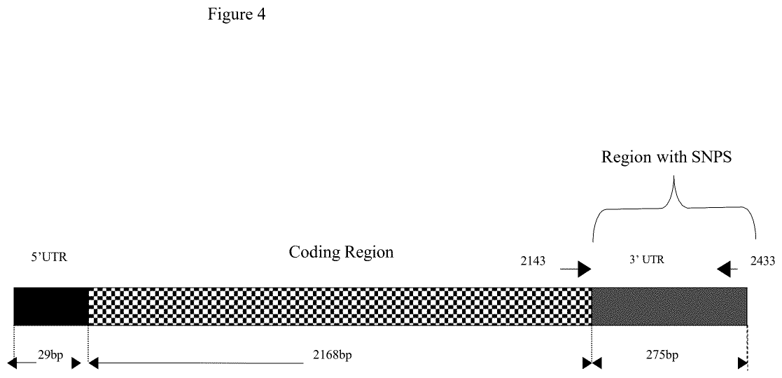

FIG. 4 shows the structure of ARNT2 mRNA, highlighting the 3′ untranslated region (UTR) that contains identified single nucleotide polymorphisms (SNPs). This figure also marks the region covered by PCR primers used for sequencing, providing insight into the specific areas targeted for genetic analysis to reveal variations associated with wound healing efficacy. The 3′ UTR ranges from base number 2169 to 2443. With real Time PCR primers designed inner part of the gene to capture the whole of the 3′UTR.

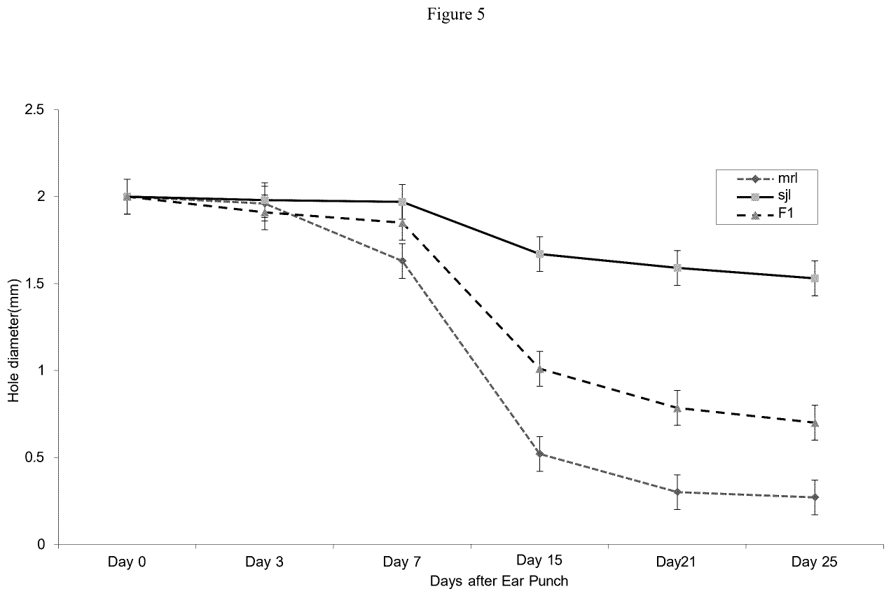

According to various embodiments of the disclosed subject matter, FIG. 5 provides a line graph illustrating the rate of ear wound healing in the parent models, MRL (good healer) and SJL (poor healer), compared to their F1 offspring, which were a cross between the two. The healing process was monitored from 24 hours to 28 days. The F1 offspring exhibited a wound healing rate that was intermediate between the good healer (MRL) and the poor healer (SJL), suggesting a combination of genetic traits from both parent models. This indicates that while the good healer genes are passed on, the poor healer genes also influence the wound healing rate, preventing the F1 offspring from healing as rapidly as the good healer.

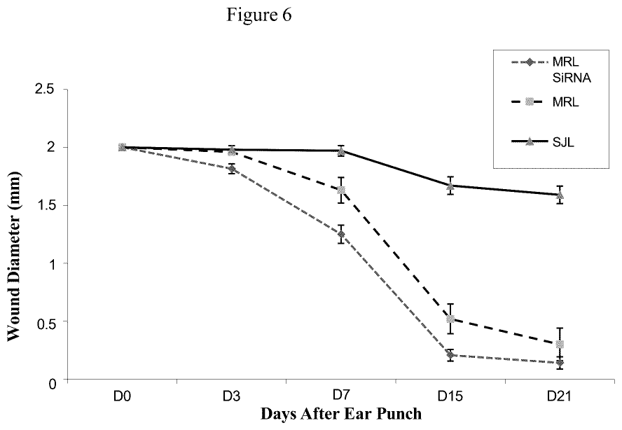

FIG. 6 is a line graph depicting the rate of wound healing in good healers and poor healers, compared to good healers treated with ARNT2 siRNA from the 3′ UTR. The good healers (MRL) and poor healers (SJL) were ear-punched, and ARNT2 siRNA was topically applied to the wounds of one set of MRL, with the other MRL and SJL groups serving as controls. The healing process was monitored from 24 hours to 21 days. The results demonstrate that the siRNA-treated wounds in the good healer group had faster wound closure and reduced scar formation compared to untreated good healers and the poor healer group. In FIG. 6, error bars represent+2 standard error of the mean (SEM).

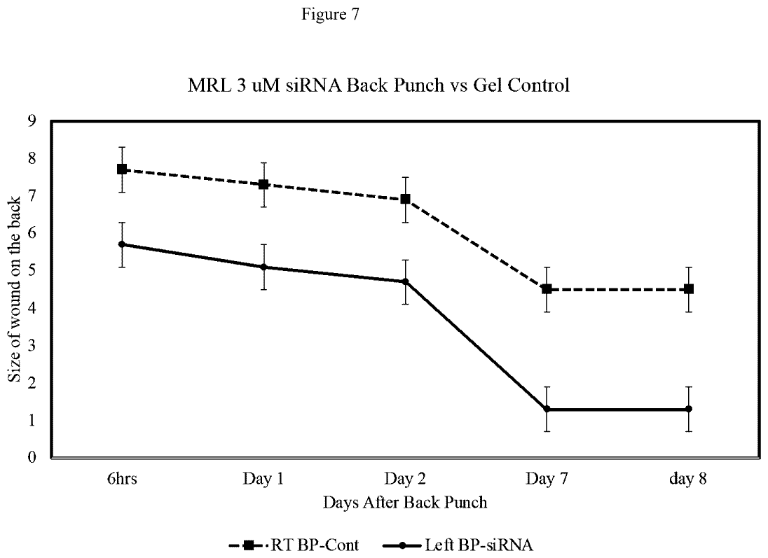

As set forth herein, FIG. 7: provides a line graph illustrating wound healing rates in the MRL mouse model (good healers) with wounding on the back. ARNT2 siRNA from the 3′ UTR was applied topically to the wound on the right side of good healers (MRL), while another left side of MRL mice served as control and were treated with gel only. The healing process was monitored from 6 hours to 8 days. The siRNA-treated wounds in the good healers demonstrated faster healing and reduced scar formation compared to the control wounds, as shown by line graphs displaying the progression of wound closure in both treated and untreated groups. In FIG. 7, error bars represent+2 SEM.

The amino acid (SEQ ID NO: 15) NM_014862 and nucleotide (SEQ ID NO: 16) M_014862 sequences of human ARNT2 are provided herein. These sequence, or any portion thereof, may be those targeted by the subject siRNAs. The subject siRNAs can bind to any 40 or fewer, 30 or fewer, 25 or fewer, 20 or fewer, 15 or fewer, 10 or fewer, or 5 or fewer, nucleotides and/or amino acids of the sequence provided by SEQ ID NO: 15 and SEQ ID NO: 16. Each amino acid of the binding region can be sequential in the sequence. Specifically, SEQ ID NO: 15 provides GenBank ID: NM_014862, which is the Protein sequence for ARNT2. In turn, SEQ ID NO: 16 provides GenBank ID: NM_014862, which is the DNA sequence for ARNT2. They are provided as follows:

Homo sapiens aryl hydrocarbon receptor nuclear translocator 2 (ARNT2), amino acids: NM_014862 (SEQ ID NO: 15): The disclosed sequence (SEQ ID NO: 15) represents the full-length amino acid sequence of the Homo sapiens ARNT2 protein. SEQ ID NO: 15 is derived from the mRNA transcript NM_014862 and provides the complete structural and functional representation of the ARNT2 protein.

MATPAAVNPPEMASDIPGSVTLPVAPMAATGQVRMAGAMPARGGKRRSG
MDFDDEDGEGPSKFSRENHSEIERRRRNKMTQYITELSDMVPTCSALAR
KPDKLTILRMAVSHMKSMRGTGNKSTDGAYKPSFLTEQELKHLILEAAD
GFLFVVAAETGRVIYVSDSVTPVLNQPQSEWFGSTLYEQVHPDDVEKLR
EQLCTSENSMTGRILDLKTGTVKKEGQQSSMRMCMGSRRSFICRMRCGN
APLDHLPLNRITTMRKRFRNGLGPVKEGEAQYAVVHCTGYIKAWPPAGM
TIPEEDADVGQGSKYCLVAIGRLQVTSSPVCMDMNGMSVPTEFLSRHNS
DGIITFVDPRCISVIGYQPQDLLGKDILEFCHPEDQSHLRESFQQVVKL
KGQVLSVMYRFRTKNREWMLIRTSSFTFQNPYSDEIEYIICTNTNVKQL
QQQQAELEVHQRDGLSSYDLSQVPVPNLPAGVHEAGKSVEKADAIFSQE
RDPRFAEMFAGISASEKKMMSSASAAGTQQIYSQGSPFPSGHSGKAFSS
SVVHVPGVNDIQSSSSTGQNMSQISRQLNQSQVAWTGSRPPFPGQQIPS
QSSKTQSSPFGIGTSHTYPADPSSYSPLSSPATSSPSGNAYSSLANRTP
GFAESGQSSGQFQGRPSEVWSQWQSQHHGQQSGEQHSHQQPGQTEVFQD
MLPMPGDPTQGTGNYNIEDFADLGMFPPFSE

Homo sapiens Aryl Hydrocarbon Receptor Nuclear Translocator 2 (ARNT2), mRNA: NM_014862, 6523 bp mRNA (SEQ ID NO: 16):

The ARNT2 gene encodes the Aryl Hydrocarbon Receptor Nuclear Translocator 2, a protein involved in various biological processes, including cellular response to environmental stimuli and regulation of gene expression. The disclosed sequence (SEQ ID NO: 16) represents the complete mRNA transcript of ARNT2, which spans 6523 base pairs.

| | |
|---|---|
| 1 | GCCGCCCGCC CGCGCCGTCC TTTGTGTGGC GGCGGCGGCG CCTGGGCCTG ACCGGGTCCC |
| 61 | CGGGGCTGAG CGCCGGGCTC CGCGCCGCCC CTCCCGCGCC CCTGCCAAGC GGGCGCCTAT |
| 121 | CCTCTCCGAG CAAGATGGCA ACCCCGGCGG CGGTCAACCC TCCGGAAATG GCTTCAGACA |
| 181 | TACCTGGATC TGTGACGTTG CCCGTTGCCC CCATGGCGGC CACCGGACAG GTGAGGATGG |
| 241 | CGGGGGCCAT GCCTGGGCGT GGAGGAAAGC GGCGTTCCGG AATGGACTTC GATGATGAAG |
| 301 | ATGGTGAAGG CCCCAGTAAA TTTTCAAGAG AGAATCATAG TGAAATCGAA AGGCGCAGAC |
| 361 | GGAACAAGAT GACTCAGTAC ATCACGGAGC TCTCCGACAT GGTCCCCACA TGCAGCGCAC |
| 421 | TGGCTCGGAA GCCAGACAAG CTCACCATCC TCCGCATGGC CGTCTCGCAC ATGAAGTCCA |
| 481 | TGAGGGGTAC AGGGAACAAG TCCACCGATG GCGCGTACAA GCCTTCCTTC CTCACAGAGC |
| 541 | AGGAACTGAA GCATCTCATC CTTGAAGCAG CTGATGGATT TCTGTTTGTG GTGGCTGCTG |
| 601 | AGACAGGGCG AGTGATTTAT GTGTCTGACT CCGTCACCCC TGTTCTGAAC CAGCCCCAGT |
| 661 | CAGAGTGGTT TGGGAGCACA CTGTATGAAC AGGTGCATCC TGATGACGTG GAGAAGCTGA |
| 721 | GAGAGCAACT GTGCACCTCA GAAAACTCAA TGACAGGCCG GATCTTGGAC CTGAAGACTG |
| 781 | GGACGGTCAA GAAAGAAGGG CAGCAGTCAT CCATGAGGAT GTGCATGGGC TCGCGGCGGT |
| 841 | CTTTCATCTG CAGGATGAGG TGTGGAAATG CTCCTTTGGA CCACCTTCCT CTAAACAGAA |
| 901 | TAACCACCAT GAGGAAAAGG TTCAGGAATG GCCTTGGCCC TGTGAAAGAA GGAGAAGCCC |
| 961 | AATATGCTGT GGTCCACTGT ACAGGATACA TCAAGGCCTG GCCACCAGCA GGAATGACCA |
| 1021 | TACCTGAAGA AGACGCTGAT GTGGGACAAG GCAGTAAATA TTGCCTCGTG GCAATTGGGA |
| 1081 | GACTCCAGGT GACCAGCTCT CCTGTATGCA TGGACATGAA TGGGATGTCG GTGCCCACAG |
| 1141 | AGTTCTTATC CCGGCATAAC TCCGATGGAA TCATCACATT TGTGGATCCA AGATGTATCA |
| 1201 | GTGTGATTGG CTACCAACCC CAGGATCTTC TGGGAAAGGA CATTTTGGAA TTCTGCCACC |
| 1261 | CTGAGGATCA AAGCCATCTG CGTGAGAGCT TCCAGCAGGT GGTTAAGCTG AAAGGCCAAG |
| 1321 | TCCTGTCGGT CATGTATCGA TTTCGCACCA AGAACCGGGA GTGGATGTTG ATCCGCACCA |
| 1381 | GCAGCTTCAC ATTCCAGAAT CCCTATTCTG ATGAGATTGA GTACATCATC TGCACCAACA |
| 1441 | CCAACGTCAA GCAACTTCAG CAACAGCAGG CAGAATTGGA AGTGCACCAG AGAGATGGAT |
| 1501 | TGTCATCGTA TGACTTATCC CAGGTCCCCG TCCCCAACCT ACCAGCCGGT GTTCATGAGG |
| 1561 | CCGGGAAGTC CGTGGAAAAG GCGGATGCAA TCTTCTCCCA GGAAAGAGAT CCTCGGTTTG |
| 1621 | CTGAAATGTT TGCAGGAATT AGTGCATCGG AGAAGAAGAT GATGAGCTCA GCCTCTGCAG |
| 1681 | CAGGAACCCA GCAGATCTAC TCCCAAGGAA GCCCATTTCC CTCTGGACAC TCCGGGAAGG |
| 1741 | CCTTCAGCTC TTCAGTGGTT CATGTGCCTG GAGTGAATGA TATTCAGTCC TCTTCTTCCA |
| 1801 | CGGGCCAGAA CATGTCCCAA ATCTCCCGGC AGCTAAACCA GAGTCAGGTG GCATGGACAG |
| 1861 | GGAGTCGTCC GCCCTTTCCG GGACAGCAAA TCCCATCTCA GTCCAGCAAG ACTCAGTCAT |
| 1921 | CTCCCTTTGG GATTGGAACG AGCCACACCT ACCCGGCAGA CCCCTCTTCC TACAGCCCCC |
| 1981 | TCTCCAGCCC AGCTACCTCC TCGCCAAGTG GGAATGCCTA CTCCAGTCTT GCCAACAGGA |
| 2041 | CTCCAGGGTT CGCTGAAAGT GGACAAAGTA GCGGGCAGTT CCAAGGGCGG CCCTCGGAAG |
| 2101 | TCTGGTCGCA GTGGCAAAGC CAGCACCATG GCCAGCAGAG CGGTGAGCAG CACTCCCACC |
| 2161 | AGCAGCCCGG TCAGACTGAA GTGTTCCAGG ACATGCTGCC CATGCCAGGA GATCCAACCC |
| 2221 | AGGGGACTGG CAACTATAAC ATCGAAGACT TTGCCGACCT GGGCATGTTT CCACCGTTTT |
| 2281 | CTGAGTAGCT GCGGCCAGAG TGAGGCTCCT GCTGTACAGT GCCACCCATA CTGTGATGTC |
| 2341 | GATGCCCATG TGAATGAGGC CCACCCTCGC CCTGCTTGCC CTGCCGCAGG CCCCCCACCA |
| 2401 | GAAGCCATCT CCCCCGCTGT GTGTCCCCAG GCGCATCATT GCTCCACTCT CCCCTGCAGC |
| 2461 | CGCGGCTGCT CGGCCACTGA CCAGGGGCCC TGGCAGACAT TAGGGGATCA GTTGTATTTA |
| 2521 | TTGTATTTTA TCTGTGTGTG CTCAGGAGGT GAGCCTAGTG GGTCCTTAAG TTTGGTTGTG |
| 2581 | AATAATCTCT TGATAGACAA TTTATACCCA TGAAAGTTCA GCCAATGCCC AGAGTGACCA |
| 2641 | AGCAGCACCA GCAGGCCTGC CCAGGATGCT GAAAGCAGAT TTCTTCTTGA TTTCACTTCT |
| 2701 | GATGGATGCA TGGGGCTCTG GGGGAATGCT GGGAAAATGG GCAAGCAGAG TGCACAGCCC |
| 2761 | TCGTTCTGCC AGCTGGAGCC AAGAAGCACA GAGACACAGA GGCGCCTGCC CCTCAGCTCC |
| 2821 | GCTGGTCTGT CTGTGCATCC ACTGTGCCGT GGGACATATG TAATTTGTGC AGTGTGACAT |
| 2881 | GGACTTGCTG TCCTAATGGA TGTGTTGTGC CTTGTGTCTG TCATGTGTGG CCAGCGTGAT |
| 2941 | TGTATAGAAT CCAGAGTATG TAGAGAGCCA AAATGTGTGG CCCTGTCCCC AAGCTCAGCA |
| 3001 | CCACCGGCCA TGACCAGAGG TTGCTCGGCC CAGGGTCTGG GAGCTGGGCT AACAGGCTGA |
| 3061 | CACTTCGGCA GGGTCACCAG GAGGCACAGC TAGGGGCATG CTCACATGGG TAGGTCAGTT |
| 3121 | CTTTGGTCCA AGCCTCTGGT GCAGATTCAA GCTCCTCCTC TTAGAGGAAG CAGCTCCTTC |
| 3181 | CTCCCACAGG AGCCCCAAGG GAGCACTCAC CCTCCTCCCA ATTGGATCTG AATAGCTGTG |
| 3241 | TTCCTGATCC CACATAAGCA TGTCTCATCC TTGCCCCACT GGTGAGAGGA GGACCTTGTG |
| 3301 | CCTGCCCAGA GCCTGCAGGA CAGCACGCCT TGAGGTCAGA GGCCTGGCCC TTCTTTACAG |
| 3361 | ATGTAGATAG CAGGGTAGGA CGCCCAGGCC TTCTCACATG GGCACCATGA AACACTTTCT |
| 3421 | TCCAGGCCAC CTGAGCCTTC GTTCTTCAGC CCGACTGGAG GAACAGCAGC TCTGGGAGGC |
| 3481 | ACAGAGTGCA GGTCTCAGGG GCAGAAGACA TGGGTTCCAG TTCTGGTTCT GCCACACACC |
| 3541 | CACCGGCCTT GAATTTCTCT GGTGGTCTCA AGACAAGAAC AAGAGCTGAC TCTAGAATCT |
| 3601 | TCTGCCTGGC TGACCTCTGA GCCCATGATC CCTGGCTTGC TGTGCTTCTC TTGGATTTGA |
| 3661 | GGAGAGTTTA TGAATCCATC CCCCTGTTTT CAGGGACCTC TGCCTTCTCC CACACCCCTT |
| 3721 | ACACTAGTGA GGACTCCTTC GGCCTCTTGT GTTTCCTTCC TTTTGTGACC ATTACGCCCC |
| 3781 | CTCCCTGTTT CTTATCACCA GCCCATCAGC CCACTGACTC CCATATGGTA GAAAAGGTAC |
| 3841 | CGTGGCTGCT GCAGGGCACA TGGAGGGACC TACCCTCTGG GAAAGCCCTT GTGTCCCTCC |
| 3901 | CAGAGTCCTT CAGGTAGGCC CTCGCAGATG CTGCTTGCTC AGAAACAACC CGTGCATCCC |
| 3961 | TGCCCATGGA TTCTGCAGGA CCTAGAAGAG TGGCATTGGT TCTAGGAACC TGAGAGTGGA |
| 4021 | GTGAGGCCAG CATGCAGGAA GGAGTTTGGA GAGAGTGTTT GCTGTGATCC ATTGAAAGCT |
| 4081 | CCACATTAAA CCCCAGCCCT GTCTGGCCAG GGAGATCCAT GTGCTCAATG TTGCTGGGGG |
| 4141 | ACCTACAAAA GTCAAACATT GCTCAGTAAA ATGCCTTTGG CTGGTCGCCA GCTGTGCATG |
| 4201 | ACTGCAGTTC CTAGACTCTA ACTTAACCCC AACTTTGCAG TTTCTAGAGC TTTTTGTCTG |
| 4261 | TAATGGTGAT TGGGTCTCCA CTGTAGATGC ATGCAGTGTT CACTCCAACC TCAGAGGCTT |
| 4321 | TCTTGCTTCT GGGGAAATGG CAGGTGGTTG AAACCCTGCA CATCCTCCCA GGGCCAGCCC |
| 4381 | CAGAATGGGC CTTTCTCTGC CTGCCCTGCC CTTGGGGCCA CCTCTGCCTC CAGCAGGCTT |
| 4441 | GAGCTTGCCC CAGGCCTTGG GCATAATTAT TTCCCCCCAA AAAAGGCACA AAAATAGGTT |
| 4501 | TTGGATCAAA GAAGTGTTTC TCAGTGAAAT GAAAACAGTC TTTTTATAGC CTTTAGCTTG |
| 4561 | TGAGTTTGGA AGTTTGGGGG GTCTTATGTT TGTTTTGCCT CTTCTGTTTC TTGGAGGAGA |
| 4621 | GTTGAGGCTT TTCTTAGGTG CATACACAGA CCCAGGTGAA CACGCTGACT GTGAACCTGC |
| 4681 | CCTGTATCCG GAGCTGTGCT GGGCACTGAG GGGATGCAAC AAAATTAGGA GAGGATCCTT |
| 4741 | GCTCCCAACG TCTACTTCTC CTACCTCAAC AGGGGTCCAG GGTGCAGTGA ACTCAGTTCT |
| 4801 | TGGCCCTTGG GTGAGGATTC ATGGATGAAT GAAAGCTAGA CCTGATGGGG AGGCATTATG |
| 4861 | ACTAAATAGG CCCAGCCTCC TTCCCTTCCA GCTCTGTCCT AGGAGCATAG GCGGGAAATC |
| 4921 | TGAGTAGAGT CTGACTGCAG TTTTTGCTTA TGATTTGTAA AAGCCGTCAT GGGGTCAATA |
| 4981 | AGAAAATAGG GGTGATGGAG GGGGAGAAGC CCAGGACTGG GAGAATCGCA CATGCCCCAG |
| 5041 | GGGTTTTCAC CAAGGATTTT CAAGACAAAC TGGAGTAAGA ATTAAAGCCC CAGAGGATTT |
| 5101 | AATTATCCTG GTTTGCAAAA GAGCCTCCCA TGCCAGTACC GCCCAGCCTT GGAGGCCGGA |
| 5161 | ATGCTCATGG CCCCTGTGGT CTGCTTGTCC TTCAGCCCAT GCCCAGCAGA TACCTCTCTG |
| 5221 | ACTGGAGACG GGCTCAAAGC TGGATTAGAA AGGGGAGAGG CACTTGTGAC TTTGTTTGAC |
| 5281 | TCTGTGACTC ACTTCCTCGC TCACACCTTG TTTGAACTAC TGGACTTTCA ACTGGCTTTC |
| 5341 | CTTAGGTCAG GCAAGCAGAC AGCTCCCCAC TGAAGAGGTC TGTACAGTGA CAACCCGGGC |
| 5401 | CGGCAGCAAG GACACAGATG CAGCCACAGT AAGGCTCCAT CAGGACTGGG TCAGTGATGG |
| 5461 | CAACAGGATG GCCAAGGATG GCTCTAGAAC ACTCTGTCCA TGCGTCACTC CCCCCAGTTT |
| 5521 | TATTTTTAGC TTTGGCTTCA GGGAGTGACA GCCATCACAA ATAGCCACAT TCTGCTCTAC |
| 5581 | TCTCCAACAT ACCAGATTCT ACACTGTTGT TATTTCATGA GACGTGAATG TTGCAGAGAG |
| 5641 | TGGGGGGATT CTGGTTGTTA AGGAACTTAC ACTGGGGAGC TTTACTCTTC CGTGTCAACA |
| 5701 | ATGTGACTAC ATGTTCTCCA GATTAGCCAC ACATGCAAAC ATCAGTGTCC TTCTAGCTTT |
| 5761 | AGCCGAGAAA GAAACCAGTC CCAGGGAATG AATGGTGGTC TCCCCACTCC CGGCAGCACT |
| 5821 | TTAGGCAGCC CATAAGCTAT GCGAGAATGT GAACGCTCAC CTTGCTCCGT CACGGTTCTG |
| 5881 | ACCTACCACA TAAACAGGAA GAAGCCAGTG ACCGGAACAG CTCTAGGAAT AACAAGTCAG |
| 5941 | AATAGAAGTG TCCTTTATAT TACCAGAAAA TATGGGCTTG GCCTAAGTCG CTGTCTCCTA |
| 6001 | ACCTGCCGGG GTCATTCCCC ACCAAACACC CCATACTAAG GAGCCATGAG CCACCTGGAC |
| 6061 | ATTCACCTTT TCTTTGACCA TCTGGAGTCT GGGGCAACTT AAGGAGGCAC CACACAGTGG |
| 6121 | TGCAGGCACA TTTCCAAGCG TAGGTGTCCC TGGCTTTTGT GGCCAAAGCT AGTGTTATGG |
| 6181 | TCAACAACAG GCCAGGGTCT GTGGGGCACT GACCTTGAAA GTGGCAAAAT GGAGGTTTCA |
| 6241 | CAGGCTGTGC GGGAGCAGGA CGGCTTGCTT CATCTAACAA TCTCAGTTTC CTTTAAAAAA |
| 6301 | AGAAAGAAAG GAAAAGATTT CATAAGCAGG TGTCAGTGGA CAGTTTAAGT ACTTAACCAT |
| 6361 | TTCTCTTTCT TCTTATGGAT GTGAACTGTG CTGTGGATAA ATCATTTGTA TTTCTTGAAT |
| 6421 | GTTCTCTATG ACTAACAGTT ATTAAGTCGG TTGTGTATAT GTGTAACTAA TGTAACTGCC |
| 6481 | TTTTAAAATT TCATTACAAT AAAAATGACT TTGCTCTGAA CAA |

This mRNA serves as the target of small interfering RNA (siRNA) molecules specifically designed to downregulate the expression of the ARNT2 gene. The siRNA is engineered to bind to the 3′ untranslated region (3′ UTR) of the ARNT2 transcript, mediating RNA interference (RNAi) to reduce ARNT2 protein levels effectively. The targeted downregulation of ARNT2 has applications in therapeutic contexts, including but not limited to wound healing and tissue regeneration.

For therapeutic use, a composition, and/or a cell including such system or modified by such system as disclosed herein is combined with, e.g., complexed with, a pharmaceutically acceptable carrier. The term “pharmaceutically acceptable” as used herein can refer to those compounds, materials, compositions, e.g., buffers, carriers, and/or excipients, and/or dosage forms which are, within the scope of sound medical judgment, suitable for use in contact with the tissues of human beings and/or animals without excessive toxicity, irritation, allergic response, or other problem or complication, commensurate with a reasonable benefit-to-risk ratio.

Pharmaceutically acceptable carriers, or non-pharmaceutical carriers as can be employed with the subject matter herein, include any of a phosphate buffered saline solution, water, emulsions, e.g., such as an oil/water or water/oil emulsions, and/or various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA (1975). Pharmaceutically acceptable carriers include buffers, solvents, dispersion media, coatings, isotonic and absorption delaying agents, or the like, that are compatible with pharmaceutical administration.

In some embodiments, a composition set forth herein, e.g., a pharmaceutical composition, includes a salt, e.g., NaCl, MgCl2, KCl, MgSO4, etc.; a buffering agent, e.g., a Tris buffer, N-(2-Hydroxyethyl) piperazine-N′-(2-ethanesulfonic acid) (HEPES), 2-(N-Morpholino) ethanesulfonic acid (MES), MES sodium salt, 3-(N-Morpholino) propanesulfonic acid (MOPS), N-tris [Hydroxymethyl]methyl-3-aminopropanesulfonic acid (TAPS), etc.; a solubilizing agent; a detergent, e.g., a non-ionic detergent such as Tween-20, etc.; a nuclease inhibitor; or the like. For example, in some embodiments, a subject composition includes a subject DNA-targeting RNA, e.g., gRNA, and a buffer for stabilizing nucleic acids.

In some embodiments, a subject composition, e.g., a pharmaceutical composition, contains formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetra acetic acid, “EDTA”); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, such as sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In some embodiments, a composition set forth herein, such as a pharmaceutical composition, may contain nanoparticles, e.g., polymeric nanoparticles, liposomes, or micelles (See Anselmo et al. (2016) Bioeng. Transl. Med. 1:10-29). In some embodiments, the compositions include an inorganic nanoparticle. Exemplary inorganic nanoparticles include, e.g., magnetic nanoparticles, e.g., Fe3MnO2, or silica. The outer surface of the nanoparticle can be conjugated with a positively charged polymer (e.g., polyethylenimine, polylysine, polyserine) which allows for attachment, e.g., conjugation or entrapment, of payload. In some embodiment, the pharmaceutical composition includes an organic nanoparticle, e.g., entrapment of the payload inside the nanoparticle. Exemplary organic nanoparticles include, e.g., SNALP liposomes that contain cationic lipids together with neutral helper lipids which are coated with polyethylene glycol, “PEG”, and protamine and nucleic acid complex coated with lipid coating. In some embodiment, the pharmaceutical composition includes a liposome, for example, a liposome disclosed in International (PCT) Application Publication No. WO 2015/148863.

In some embodiments, the subject compositions include a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In some embodiments, a composition, e.g., pharmaceutical composition, includes a fusogenic or endosome-destabilizing peptide or polymer.

In some embodiments, a composition as set forth herein, a pharmaceutical composition, may contain a sustained- or controlled-delivery formulation. Techniques for formulating sustained- or controlled-delivery means, such as liposome carriers, bio-erodible microparticles or porous beads and depot injections, are included in the subject disclosure. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly(2-hydroxyethyl-inethacrylate), ethylene vinyl acetate, or poly-D(-)-3-hydroxybutyric acid. Sustained release compositions may also include liposomes that can be prepared by any of several methods.

### Methods

The subject disclosure also includes a set of methods including for promoting cell promotion, such as promoting in vivo cell proliferation. The proliferation can include promoting tissue growth, such as tissue regrowth wherein the regrowth occurs at a subject wound site. Embodiments of the methods include administering an amount, e.g., a therapeutically effective amount, of a subject composition, such as a composition including a small interfering RNA (siRNA). Such an siRNA may in turn be configured to target a 3′ untranslated region (UTR) of ARNT2 gene of a subject and thereby decrease expression of ARNT2 in the subject compared to what such expression would be in the absence of such a composition.

The methods may include providing rapid would healing and/or tissue generation and/or tissue regeneration for a subject. Applying the subject compositions may include promoting wound healing by enhancing tissue regeneration and/or reducing scar formation.

ARNT2 is a member of the basic helix-loop-helix-PAS (bHLH-PAS) family of proteins, plays a significant role in regulating gene expression during cellular responses to hypoxia, a condition commonly associated with wound healing. Accordingly, the subject methods include modulating, such as by down-regulating, expression of the ARNT2 gene and thereby promoting wound healing.

The presently disclosed methods include the identification and/or design, as well as the application of specific siRNA sequences associated with the 3′ UTR of the ARNT2 gene, e.g., siRNA designed to target the 3′ UTR of an ARNT2 mRNA transcript as shown, for example, by SEQ ID NO: 17. By binding to this UTR region, the siRNA triggers the RNA-induced silencing complex (RISC) to degrade the ARNT2 mRNA, effectively reducing its translation into functional ARNT2 protein. This down regulation of ARNT2, such as those formed with hypoxia-inducible factor 1-alpha (HIF-1 alpha). The siRNA molecules were designed based on optimal sequences that exhibit high specificity and minimal off target effects, disrupts the formation of transcriptionally functional ARNT2.

ARNT2 modulates the expression of various genes involved in the cellular response to hypoxia, including those that regulate angiogenesis, inflammation, and extracellular matrix remodeling. By silencing and/or minimizing expression of ARNT2 according to the subject methods, the methods include altering the hypoxic response pathways of a subject, promoting a wound healing environment that favors rapid tissue regeneration without excessive fibrosis.

The mechanism of action according to the subject embodiments can be described in three stages: an initial response stage, downregulation of ARNT2, and enhanced tissue repair. During the initial response stage, upon injury, siRNA molecules targeting 3′ UTR of ARNT2 gene are administered topically at the wound site. These siRNAs are taken up by the cells in the wound environment, where they bind to ARNT2 mRNA and initiate degradation. During the downregulation of ARNT2 stage, the reduction in ARNT2 protein levels affects the transcription of hypoxia-regulated genes, which in turn decreases inflammation, promotes angiogenesis, and enhances cellular proliferation and migration. During the enhanced tissue repair stage, with the ARNT2 downregulated, the repair process proceeds with reduced scar tissue formation. The matrix remodeling is more efficient than in the absence of the subject compositions, and the wound heals more rapidly. By downregulating ARNT2 expression, the subject methods enhance the natural regenerative capacity of tissues, mimicking the scarless wound healing observed in amphibians.

The presently disclosed subject matter leverages ARNT2's role in the regulation of hypoxia-responsive genes and transcriptional activities, particularly those involved in cell migration, proliferation, and tissue remodeling. The subject methods utilize siRNA molecules to specifically target and silence or minimize ARNT2 expression, leading to improved wound healing by accelerating tissue repair processes, reducing inflammation, and/or preventing fibrotic scar formation.

According to some versions, the methods include administering a composition topically to a wound site of the subject. In one embodiment, siRNA molecules are delivered topically in the form of gel, cream, Accell self-delivering siRNA or hydrogel, in a manner providing direct contact with the wound bed. The administering may also be performed by injecting the siRNA into the subject. Such injection may be intravenous or subcutaneous. It may also be systematic, such as via intravenous therapy, such as directly into a subject's venous system.

In some versions, the methods include administering by applying a delivery methodology, e.g., vehicle, selected from: nanoparticles, liposomes, hydrogels, self-delivering siRNA, another pharmaceutical carrier, or any combination thereof. Encapsulating siRNA compounds as set forth herein in delivery vehicles such as nanoparticles or liposomes can include enhancing stability, protecting the siRNA from degradation, and/or promoting cellular uptake. Such delivery vehicles can be designed for both localized and systemic administration, depending on the nature and extent of the wound.

In some versions, the methods include diagnosing and/or treating an injury or disorder, such as treating the disorder based on the diagnosis. The injury or disorder may be a skin disorder. The injury or disorder may be a wound site in a subject. The wound site may be a surgical, traumatic, chronic, or burn wound site. The disorder may also be aged skin, such as skin that is thin, torn, discolored, and/or or exhibits keratosis or scarring. Aging skin is characterized by, e.g., reduced collagen production, impaired wound healing and/or loss of elasticity due to oxidative stress and decreased cellular activity. ARNT2 modulates several key processes like reduced hypoxia response, decline in extracellular matrix remodeling, impaired cellular proliferation, and oxidative stress regulation to reverse the aging process.

The subject embodiments include applying the subject compositions and/or thereby treating, acute wounds, e.g., wounds resulting from surgery, trauma, or burns, where rapid healing without scarring is desired; and/or chronic wounds, e.g., diabetic ulcers or pressure sores, where conventional healing processes are slow or impaired. Subject embodiments also include applying the compositions for cosmetic applications, such as post-surgical or dermatological procedures where minimizing scarring is a priority. Embodiments of the methods also include treating conditions where hypoxic gene regulation plays a role, such as ischemic tissue injuries.

The subject methods also include administering growth factors, anti-inflammatory agents, or matrix metalloproteinase inhibitors to the subject. The methods can also include decreasing inflammation, increasing cell migration, and improving extracellular matrix remodeling in the subject.

Aspects of the subject methods also include methods of manufacturing a cell proliferation composition, such as any composition described herein by, generating a cell proliferation composition, e.g., a composition comprising a small interfering RNA (siRNA) targeting a 3′ untranslated region (UTR) of an ARNT2 gene of a subject, and packaging the cell proliferation composition.

Embodiments of the methods also include identifying a candidate compound for treatment of a wound site of a subject by, for example: contacting a sample comprising an ARNT2 gene with a test compound comprising a small interfering RNA (siRNA) targeting a 3′ untranslated region (UTR) of the ARNT2 gene; determining if the test compound decreases ARNT2 expression level in the sample; and/or selecting the test compound as a candidate if the ARNT2 expression level is decreased.

A composition as set forth herein can be administered by a variety of methods. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration, e.g., by injection or infusion. Depending on the route of administration, the active compound, e.g., the guide nucleic acid, engineered, non-naturally occurring system, e.g., vector pool, disclosed herein, may be coated in a material to protect the compound from the action of acids and other natural conditions that may inactivate the compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline, “PBS”. The carrier should be stable under the conditions of manufacture and storage, and should be preserved against microorganisms. The carrier can be a solvent or dispersion medium containing, for example, water, ethanol, polyol, for example, glycerol, propylene glycol, and liquid polyethylene glycol, and suitable mixtures thereof.

Pharmaceutical formulations can be sterile. Sterilization can be accomplished by any suitable method, e.g., filtration through sterile filtration membranes. Where the composition is lyophilized, filter sterilization can be conducted prior to or following lyophilization and reconstitution. In some embodiments, the pharmaceutical composition is lyophilized, and then reconstituted in buffered saline, at the time of administration.

Pharmaceutical compositions of the subject matter disclosed herein can be prepared in accordance with various methods as set forth, for example in: Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions can be manufactured under GMP conditions. The compositions disclosed herein are formulated into pharmaceutically acceptable dosage forms by various methods. Dosage regimens are adjusted to provide the optimum desired response, e.g., a therapeutic response. For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions disclosed herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### Kits

Variations of the subject embodiments include kits for promoting cell proliferation, such as cell growth and/or regrowth, such as tissue growth and/or regrowth. The kits can include any of the aspects described herein such as small interfering RNAs (siRNA) targeting a 3′ untranslated region (UTR) of an ARNT2 gene of a subject including one or more siRNAs; a buffer solution; and/or packaging.

In various aspects, the subject kits include any composition or combination of compositions or aspects thereof and packaging. In various instances the packaging is configured to retain the compositions therein for shipping and/or storing. The packaging can include plastic, rubber, fabric, cardboard, paper, styrofoam, glass or any combination thereof. The packaging can be a bottle or container and/or a reciprocal container cap. The packaging can include a bag and/or box and be configured for shipping and/or cell preservation.

In various embodiments of kits set forth herein, one or more of the elements of the system are provided in lyophilized form, and the kit includes a diluent. Elements may be provided individually or in combinations, and may be provided in a container, such as a vial, a bottle, a tube, or immobilized on the surface of a solid base, e.g., chip or microarray. In various embodiments, a kit includes one or more of the nucleic acids and/or cells described herein.

In various embodiments, the subject kits include one or more reagents and/or buffers for use in a process utilizing one or more of the elements described herein. Reagents may be provided in a container and may be provided in a form that is usable in a particular assay, or in a form that requires addition of one or more other components before use, e.g., in concentrate or lyophilized form. A buffer may be a reaction or storage buffer, including but not limited to a sodium carbonate buffer, a sodium bicarbonate buffer, a borate buffer, a Tris buffer, a MOPS buffer, a HEPES buffer, and/or any combinations thereof. In some embodiments, the buffer is alkaline. In various embodiments, the buffer has a pH from 6 to 11, 7 to 10, or 7 to 9. In various embodiments, the kit includes a pharmaceutically acceptable carrier. In various embodiments, the kit includes one or more devices or other materials for administration to a subject, e.g., a syringe.

### Utility

The presently disclosed subject matter includes compositions and methods for promoting rapid wound healing and tissue regeneration. The subject matter, in various aspects, applies compounds including small interfering RNA (siRNA) that target the 3′ untranslated region (UTR) of the aryl hydrocarbon nuclear transporter 2 (ARNT2) gene. As described herein, through quantitative trait loci (QTL) mapping on chromosome 7, ARNT2 was identified as a candidate gene involved in wound healing, with specific 3′ UTR sequences segregating with either good or poor wound healing outcomes. By downregulating ARNT2 expression, the subject methods modulate key pathways involved in tissue repair, including inflammation, cell migration, and extracellular matrix remodeling, and thereby result in faster wound healing with minimal scar formation.

The subject compositions and methods include the design and administration of specific siRNA sequences, either topically mixed in hydrogels, Accell technology, and use of delivery vehicles such as nanoparticles or liposomes to enhance siRNA stability and cellular uptake. Experimental results demonstrate a dose-dependent reduction in in ARNT2 expression, improved tissue regeneration, and reduced fibrosis in treated wounds. The method is applicable to various types of wounds, including acute, chronic, and surgical wounds, and offers a promising therapeutic strategy for enhancing wound healing and preventing scarring in mammals, e.g., humans.

The subject embodiments address a significant need for therapeutics that can expedite wound healing and prevent scar formation in subjects, e.g., patients, with injuries resulting from trauma, surgery, and/or chronic conditions. By harnessing siRNA from the 3′ UTR to regulate ARNT2 expression, the methods described herein provide an effective approach to enhancing tissue regeneration and improving patient outcomes.

### EXAMPLES

The following example(s) are set forth so as to provide those of ordinary skill in the art with a complete disclosure and description of how to make and use the presently disclosed subject matter and are not intended to limit the scope of what the inventors regard as their invention nor are they intended to represent that the experiments below are all or the only experiments performed. Efforts have been made to ensure accuracy with respect to numbers used, e.g. amounts, temperature, etc., but experimental errors and deviations should be accounted for. Unless indicated otherwise, parts are parts by weight, molecular weight is weight average molecular weight, temperature is in degrees Centigrade, and pressure is at or near atmospheric.

### Example 1

In an experimental model, siRNA sequences targeting the ARNT2 3′ UTR were synthesized and tested in vitro on cultured rat skin fibroblasts, key cell type involved in wound healing. Various concentrations of siRNA, ranging from 100 ng to 300 ng, were applied to determine the optimal dose for maximal ARNT2 knockdown and enhanced wound healing outcomes.

Table 2 illustrates the effect of siRNA targeting the 3′ UTR of ARNT2-on-ARNT2 expression levels in rat fibroblast skin cells. Table 2 compares the downregulation of ARNT2 at different siRNA concentrations-100 ng, 200 ng, and 300 ng-versus the control. The results indicate a dose-dependent reduction in ARNT2 expression. However, at higher concentrations (200-300 ng), ARNT2 mRNA levels were significantly reduced, indicating a dose dependent downregulation of the siRNA from the 3′ UTR. The sequence used in this study was, GUACUUUAUCUGUGUGCUC in a sense and antisense double stranded format and it was effective in lowering ARNT2 expression from 100 ng to 300 ng with lowest downregulation levels at 300 ng.

TABLE 2
siRNA Targeting 3′ UTR of
ARNT2-on-ARNT2 in Skin Fibroblasts

| Conc | Mean | | P- | |
|---|---|---|---|---|
| siRNA | Cycles | SD | value | Regulation |
| 100 ng | 32.36 | 0.094 | 0.00001 | Down |
| 200 ng | 33.33 | 0.85 | 0.0001 | Down |
| 300 ng | 36 | 0.25 | 0.00001 | Down |
| Control | 24.7 | 0.27 | | |

### Example 2

In vivo studies in good and poor wound healing mouse models further confirmed that good healers had lower levels of ARNT2 as compared to poor healers. This was demonstrated at different time points from 4 hours after wounding to 28 days, with mRNA isolated and the levels of ARNT2 in both models compared using real time PCR. Wounds treated with siRNA targeting ARNT2 from 3′ UTR healed significantly faster compared to controls, with a marked reduction in fibrotic tissue formation. Observation of the wounds showed no scarring and could not identify where the wound was previously.

### Example 3

Table 3 provides data for two mice models, MRL (good healer) and SJL (poor healer), ear-punched at 4 hours to 28 days and the expression levels of ARNT2 were monitored by real-time PCR from 4 hours to 28 days. The real-time PCR results showed lower expression levels of ARNT2 in the good healer (MRL), as indicated by fewer amplification cycles, suggesting that ARNT2 expression is significantly lower in the good healer compared to the poor healer (SJL). This downregulation of ARNT2 expression in the good healer is associated with faster wound healing and reduced scarring, suggesting a link between lower ARNT2 expression levels and improved wound healing outcomes.

TABLE 3
Healing for MRL (Good Healer) and SJL (Poor Healer)

| Time | MRL Increase | SJL Increase | |
|---|---|---|---|
| point | in cycle# | in cycle # | P-value |
| 4 hours | 2.56 ± 0.747 | 3.87 ± 0.262 | 0.04 |
| Day 1 | 4.317 ± 0.530 | 5.244 ± 0.0169 | 0.04 |
| Day 7 | 2.92 ± 0.414 | 4.85 ± 0.558 | 0.0086 |
| Day 14 | 2.19 ± 0.301 | 6.353 ± 0.116 | 0.00001 |
| Day 28 | 3.182 ± 0.834 | 5.459 ± 0.275 | 0.0109 |

### Exemplary Non-Limiting Aspects of the Disclosure

The aspects, including embodiments, of the present disclosure described above may be advantageous when utilized alone or in combination with one or more other aspects or embodiments. The following non-limiting aspects of the disclosure are provided to further clarify and support the described subject matter. As will be apparent to those skilled in the art, each of the aspects may be combined with any of the preceding or following aspects, providing support for all such combinations. This list is not exhaustive, and the combinations should not be limited to those explicitly listed below.

1. An in vivo cell proliferation composition, comprising, i. A synthetic short interfering RNA (siRNA) targeting a 3′ untranslated region (UTR) of an aryl hydrocarbon nuclear receptor translocator 2 (ARNT2), such as the sequence set forth in SEQ ID NO:17; and ii. A pharmaceutically acceptable carrier.

2. The composition of claim 1, wherein the synthetic siRNA is double-stranded.

3. The composition of claim 1 or claim 2, wherein the siRNA reduces the expression levels of an ARNT2 gene or transcript when bound to the ARNT2 gene.

4. The composition of any one of claims 1 to 3, wherein the siRNA has a length of 30 nucleotides or fewer.

5. The composition of any one of claims 1 to 4, wherein the 3′ UTR targeted by the siRNA comprises one or more single nucleotide polymorphisms (SNPs).

6. The composition of any one of claims 1 to 5, wherein the composition is formulated as a topical gel, cream, hydrogel, or self-delivering siRNA system.

7. The composition of any one of claims 1 to 6, further comprising liposomes encapsulating the siRNA and a pharmaceutically acceptable carrier.

8. The composition of any one of claims 1 to 7, further comprising pharmaceutical delivery nanoparticles.

9. The composition of any one of claims 1 to 8, wherein the 3′ UTR targeted by the siRNA has a length of 300 base pairs or fewer.

10. The composition of any one of claims 1 to 9, wherein the pharmaceutically acceptable carrier is suitable for topical administration.

11. The composition of any one of claims 1 to 10, wherein the pharmaceutically acceptable carrier is suitable for injectable administration.

12. The composition of any one of claims 1 to 11, further comprising liposomes as a delivery vehicle.

13. The composition of any one of claims 1 to 12, further comprising nanoparticles or hydrogels for delivery of the siRNA.

14. The composition of any one of claims 1 to 13, wherein the siRNA is a self-delivering siRNA.

15. The composition of any one of claims 1 to 14, wherein the siRNA is present at a concentration ranging from 100 nM to 300 nM.

16. The composition of any one of claims 1 to 15, further comprising one or more additional agents selected from growth factors, anti-inflammatory agents, or matrix metalloproteinase inhibitors.

17. A method for promoting in vivo cell proliferation, comprising i. Administering a therapeutically effective amount of synthetic siRNA targeting the 3′ UTR of the ARNT2 gene, thereby decreasing the expression of ARNT2 in a subject.

18. The method of claim 17, wherein the siRNA is administered topically to a wound site of the subject.

19. The method of claim 17, wherein the siRNA is administered by injection into the subject.

20. The method of any of claims 17-19, wherein the administering is via a delivery vehicle selected from nanoparticles, liposomes, hydrogels, self-delivering siRNA, or another pharmaceutical carrier.

21. The method of any of claims 17-20, wherein the concentration of siRNA administered is in the range of 100 nM to 300 nM.

22. The method of any of claims 17-21, wherein the administering is to a wound site selected from surgical, traumatic, chronic, or burn wound sites.

23. The method of any of claims 17-22, further comprising administering growth factors, anti-inflammatory agents, or matrix metalloproteinase inhibitors to the subject.

24. The method of any of claims 17-23, further comprising decreasing inflammation, increasing cell migration, and improving extracellular matrix remodeling in the subject.

25. A method of manufacturing a cell proliferation composition, comprising, i. Generating a cell proliferation composition comprising synthetic siRNA targeting the 3′ UTR of the ARNT2 gene; and ii. Packaging the composition.

26. A method for identifying a candidate compound for treating a wound site of a subject, comprising,
i. Contacting a sample comprising an ARNT2 gene with a test compound comprising synthetic siRNA targeting the 3′ UTR of the ARNT2 gene;
ii. Determining if the test compound decreases ARNT2 expression in the sample; and

27. Selecting the test compound as a candidate if the ARNT2 expression level is decreased.

28. A kit for promoting cell proliferation, comprising, i. Synthetic siRNA targeting the 3′ UTR of the ARNT2 gene, which may include one or more single nucleotide polymorphisms (SNPs);
ii. A buffer solution; and iii. Packaging.

29. The composition of any of claims 1-16, wherein the siRNA has at least 70% identity to a sequence selected from Table 1, or to a combination of two sequences selected from Table 1.

While the embodiments have been described in detail for illustrative purposes, it will be apparent to those skilled in the art that certain changes or modifications may be made without departing from the spirit or scope of the appended claims. The terminology used herein is for describing specific embodiments and is not intended to be limiting. The scope of the invention is determined solely by the appended claims.

The preceding illustrates the principles of the invention. Those skilled in the art will appreciate that various modifications can be devised which embody the principles of the invention, even if not explicitly described herein. Examples and conditional language are provided primarily to aid in understanding the invention's principles and should not be construed as limiting. All statements reciting aspects or principles of the invention, along with specific examples, are intended to encompass both structural and functional equivalents. These equivalents include both current and future developments that perform the same function, regardless of structural differences. The scope of the disclosed subject matter is, therefore, defined by the appended claims, not by the specific embodiments described above.

1. An in vivo cell proliferation composition comprising:
i. a synthetic short interfering RNA (siRNA) targeting the 3′ untranslated region (UTR) of an aryl hydrocarbon nuclear receptor translocator 2 (ARNT2); and
ii. a pharmaceutically acceptable carrier.

2. The composition of claim 1, wherein the synthetic short interfering RNA (siRNA) is chemically modified to increase stability in physiological conditions.

3. The composition of claim 1, wherein the pharmaceutically acceptable carrier is selected from a group consisting of liposomes, nanoparticles, micelles, or hydrogels.

4. The composition of claim 1, further comprising a transfection enhancer to improve delivery of the siRNA into target cells in vivo.

5. The composition of claim 1, wherein the siRNA is delivered in a nanoparticle formulation designed to specifically target wound or injury sites.

6. The composition of claim 1, wherein the siRNA is designed to minimize off-target effects while retaining high specificity for ARNT2.

7. The composition of claim 1, wherein the siRNA is encapsulated in a lipid nanoparticle system to improve cellular uptake and reduce immune response.

8. The composition of claim 1, further comprising a targeting ligand that directs the siRNA-carrier complex to specific tissues or cell types expressing ARNT2.

9. The composition of claim 1, further comprising an adjuvant to enhance the therapeutic effect of the siRNA.

10. The composition of claim 1, wherein the siRNA is co-formulated with growth factors or cytokines to synergistically promote cell proliferation.

11. A method for promoting in vivo cell proliferation comprising administering a therapeutically effective amount of synthetic small interfering RNA (siRNA) targeting the 3′ untranslated region (UTR) of the ARNT2 gene in a subject, thereby decreasing ARNT2 expression in the subject.

12. The method according to claim 11, wherein the administering is performed topically to a wound site of the subject.

13. The method according to claim 11, wherein the administering is performed by injecting the siRNA into the subject.

14. The method according to claim 11, wherein the administering is via delivery vehicle selected from the group consisting of: nanoparticles, liposomes, hydrogels, a self-delivering siRNA, or another pharmaceutical carrier.

15. The method according to claim 11, wherein the concentration of siRNA administered is in a range from 100 ng to 300 ng.

16. A method of manufacturing a cell proliferation composition comprising:
i. generating a composition that includes a synthetic small interfering RNA (siRNA) targeting the 3′ untranslated region (UTR) of an ARNT2 gene of a subject, and
ii. packaging the cell proliferation composition.

17. The method of claim 16, wherein the synthetic small interfering RNA (siRNA) is chemically modified to enhance stability during storage and transport.

18. The method of claim 16, wherein the composition further comprises a pharmaceutically acceptable carrier or excipient.

19. The method of claim 16, wherein the siRNA is specific to a sequence within the 3′ untranslated region (UTR) of the ARNT2 gene.

20. The method of claim 16, wherein the composition is packaged in a sterile container suitable for clinical administration.

21. The method of claim 16, further comprising testing the siRNA for its specificity and efficacy in silencing the ARNT2 gene.

22. The kit of claim 16, wherein the buffer solution is supplemented with antioxidants to prevent oxidative degradation of the siRNA.

23. The kit of claim 16, wherein the packaging is vacuum-sealed or nitrogen-purged to enhance shelf life.


SEQUENCE LISTING (35 sequences)

SEQ ID NO: 1 - RNA, 19 nt, synthetic construct
  source 1..19: mol_type = other RNA
gtactttatctgtgtgctc

SEQ ID NO: 2 - RNA, 19 nt, synthetic construct
  source 1..19: mol_type = other RNA
ccatgttcaaagagggtcc

SEQ ID NO: 3 - RNA, 19 nt, synthetic construct
  source 1..19: mol_type = other RNA
agcttgccctgctgtagaa

SEQ ID NO: 4 - RNA, 19 nt, synthetic construct
  source 1..19: mol_type = other RNA
ccccatcctccctggacat

SEQ ID NO: 5 - RNA, 19 nt, synthetic construct
  source 1..19: mol_type = other RNA
gttaagaagttgagggtaa

SEQ ID NO: 6 - RNA, 19 nt, synthetic construct
  source 1..19: mol_type = other RNA
gaactaacttctagaacaa

SEQ ID NO: 7 - RNA, 19 nt, synthetic construct
  source 1..19: mol_type = other RNA
gggcagagcaagcaggaaa

SEQ ID NO: 8 - RNA, 19 nt, synthetic construct
  source 1..19: mol_type = other RNA
gaatgagcctggagattaa

SEQ ID NO: 9 - RNA, 19 nt, synthetic construct
  source 1..19: mol_type = other RNA
gctttaaacctgagaatca

SEQ ID NO: 10 - RNA, 19 nt, synthetic construct
  source 1..19: mol_type = other RNA
gagcagaggtggaggattg

SEQ ID NO: 11 - RNA, 19 nt, synthetic construct
  source 1..19: mol_type = other RNA
ggacagctttgaagacata

SEQ ID NO: 12 - RNA, 19 nt, synthetic construct
  source 1..19: mol_type = other RNA
acctttagcttgtgaatta

SEQ ID NO: 13 - RNA, 19 nt, synthetic construct
  source 1..19: mol_type = other RNA
gggagaagtcagaaatgaa

SEQ ID NO: 14 - RNA, 19 nt, synthetic construct
  source 1..19: mol_type = other RNA
gggtagtagtatttggatt

SEQ ID NO: 15 - protein, 717 aa, Homo sapiens
MATPAAVNPPEMASDIPGSVTLPVAPMAATGQVRMAGAMPARGGKRRSGMDFDDEDGEGPSKFSRENHSEIERRRRNKMTQYITELSDMVPTCSALARKPDKLTILRMAVSHMKSMRGTGNKSTDGAYKPSFLTEQELKHLILEAADGFLFVVAAETGRVIYVSDSVTPVLNQPQSEWFGSTLYEQVHPDDVEKLREQLCTSENSMTGRILDLKTGTVKKEGQQSSMRMCMGSRRSFICRMRCGNAPLDHLPLNRITTMRKRFRNGLGPVKEGEAQYAVVHCTGYIKAWPPAGMTIPEEDADVGQGSKYCLVAIGRLQVTSSPVCMDMNGMSVPTEFLSRHNSDGIITFVDPRCISVIGYQPQDLLGKDILEFCHPEDQSHLRESFQQVVKLKGQVLSVMYRFRTKNREWMLIRTSSFTFQNPYSDEIEYIICTNTNVKQLQQQQAELEVHQRDGLSSYDLSQVPVPNLPAGVHEAGKSVEKADAIFSQERDPRFAEMFAGISASEKKMMSSASAAGTQQIYSQGSPFPSGHSGKAFSSSVVHVPGVNDIQSSSSTGQNMSQISRQLNQSQVAWTGSRPPFPGQQIPSQSSKTQSSPFGIGTSHTYPADPSSYSPLSSPATSSPSGNAYSSLANRTPGFAESGQSSGQFQGRPSEVWSQWQSQHHGQQSGEQHSHQQPGQTEVFQDMLPMPGDPTQGTGNYNIEDFADLGMFPPFSE

SEQ ID NO: 16 - RNA, 6523 nt, Homo sapiens
  source 1..6523: mol_type = mRNA
gccgcccgcccgcgccgtcctttgtgtggcggcggcggcgcctgggcctgaccgggtccccggggctgagcgccgggctccgcgccgcccctcccgcgcccctgccaagcgggcgcctatcctctccgagcaagatggcaaccccggcggcggtcaaccctccggaaatggcttcagacatacctggatctgtgacgttgcccgttgcccccatggcggccaccggacaggtgaggatggcgggggccatgcctgcccgtggaggaaagcggcgttccggaatggacttcgatgatgaagatggtgaaggccccagtaaattttcaagagagaatcatagtgaaatcgaaaggcgcagacggaacaagatgactcagtacatcacggagctctccgacatggtccccacatgcagcgcactggctcggaagccagacaagctcaccatcctccgcatggccgtctcgcacatgaagtccatgaggggtacagggaacaagtccaccgatggcgcgtacaagccttccttcctcacagagcaggaactgaagcatctcatccttgaagcagctgatggatttctgtttgtggtggctgctgagacagggcgagtgatttatgtgtctgactccgtcacccctgttctgaaccagccccagtcagagtggtttgggagcacactgtatgaacaggtgcatcctgatgacgtggagaagctgagagagcaactgtgcacctcagaaaactcaatgacaggccggatcttggacctgaagactgggacggtcaagaaagaagggcagcagtcatccatgaggatgtgcatgggctcgcggcggtctttcatctgcaggatgaggtgtggaaatgctcctttggaccaccttcctctaaacagaataaccaccatgaggaaaaggttcaggaatggccttggccctgtgaaagaaggagaagcccaatatgctgtggtccactgtacaggatacatcaaggcctggccaccagcaggaatgaccatacctgaagaagacgctgatgtgggacaaggcagtaaatattgcctcgtggcaattgggagactccaggtgaccagctctcctgtatgcatggacatgaatgggatgtcggtgcccacagagttcttatcccggcataactccgatggaatcatcacatttgtggatccaagatgtatcagtgtgattggctaccaaccccaggatcttctgggaaaggacattttggaattctgccaccctgaggatcaaagccatctgcgtgagagcttccagcaggtggttaagctgaaaggccaagtcctgtcggtcatgtatcgatttcgcaccaagaaccgggagtggatgttgatccgcaccagcagcttcacattccagaatccctattctgatgagattgagtacatcatctgcaccaacaccaacgtcaagcaacttcagcaacagcaggcagaattggaagtgcaccagagagatggattgtcatcgtatgacttatcccaggtccccgtccccaacctaccagccggtgttcatgaggccgggaagtccgtggaaaaggcggatgcaatcttctcccaggaaagagatcctcggtttgctgaaatgtttgcaggaattagtgcatcggagaagaagatgatgagctcagcctctgcagcaggaacccagcagatctactcccaaggaagcccatttccctctggacactccgggaaggccttcagctcttcagtggttcatgtgcctggagtgaatgatattcagtcctcttcttccacgggccagaacatgtcccaaatctcccggcagctaaaccagagtcaggtggcatggacagggagtcgtccgccctttccgggacagcaaatcccatctcagtccagcaagactcagtcatctccctttgggattggaacgagccacacctacccggcagacccctcttcctacagccccctctccagcccagctacctcctcgccaagtgggaatgcctactccagtcttgccaacaggactccagggttcgctgaaagtggacaaagtagcgggcagttccaagggcggccctcggaagtctggtcgcagtggcaaagccagcaccatggccagcagagcggtgagcagcactcccaccagcagcccggtcagactgaagtgttccaggacatgctgcccatgccaggagatccaacccaggggactggcaactataacatcgaagactttgccgacctgggcatgtttccaccgttttctgagtagctgcggccagagtgaggctcctgctgtacagtgccacccatactgtgatgtcgatgcccatgtgaatgaggcccaccctcgccctgcttgccctgccgcaggccccccaccagaagccatctcccccgctgtgtgtccccaggcgcatcattgctccactctcccctgcagccgcggctgctcggccactgaccaggggccctggcagacattaggggatcagttgtatttattgtattttatctgtgtgtgctcaggaggtgagcctagtgggtccttaagtttggttgtgaataatctcttgatagacaatttatacccatgaaagttcagccaatgcccagagtgaccaagcagcaccagcaggcctgcccaggatgctgaaagcagatttcttcttgatttcacttctgatggatgcatggggctctgggggaatgctgggaaaatgggcaagcagagtgcacagccctcgttctgccagctggagccaagaagcacagagacacagaggcgcctgcccctcagctccgctggtctgtctgtgcatccactgtgccgtgggacatatgtaatttgtgcagtgtgacatggacttgctgtcctaatggatgtgttgtgccttgtgtctgtcatgtgtggccagcgtgattgtatagaatccagagtatgtagagagccaaaatgtgtggccctgtccccaagctcagcaccaccggccatgaccagaggttgctcggcccagggtctgggagctgggctaacaggctgacacttcggcagggtcaccaggaggcacagctaggggcatgctcacatgggtaggtcagttctttggtccaagcctctggtgcagattcaagctcctcctcttagaggaagcagctccttcctcccacaggagccccaagggagcactcaccctcctcccaattggatctgaatagctgtgttcctgatcccacataagcatgtctcatccttgccccactggtgagaggaggaccttgtgcctgcccagagcctgcaggacagcacgccttgaggtcagaggcctggcccttctttacagatgtagatagcagggtaggacgcccaggccttctcacatgggcaccatgaaacactttcttccaggccacctgagccttcgttcttcagcccgactggaggaacagcagctctgggaggcacagagtgcaggtctcaggggcagaagacatgggttccagttctggttctgccacacacccaccggccttgaatttctctggtggtctcaagacaagaacaagagctgactctagaatcttctgcctggctgacctctgagcccatgatccctggcttgctgtgcttctcttggatttgaggagagtttatgaatccatccccctgttttcagggacctctgccttctcccacaccccttacactagtgaggactccttcggcctcttgtgtttccttccttttgtgaccattacgccccctccctgtttcttatcaccagcccatcagcccactgactcccatatggtagaaaaggtaccgtggctgctgcagggcacatggagggacctaccctctgggaaagcccttgtgtccctcccagagtccttcaggtaggccctcgcagatgctgcttgctcagaaacaacccgtgcatccctgcccatggattctgcaggacctagaagagtggcattggttctaggaacctgagagtggagtgaggccagcatgcaggaaggagtttggagagagtgtttgctgtgatccattgaaagctccacattaaaccccagccctgtctggccagggagatccatgtgctcaatgttgctgggggacctacaaaagtcaaacattgctcagtaaaatgcctttggctggtcgccagctgtgcatgactgcagttcctagactctaacttaaccccaactttgcagtttctagagctttttgtctgtaatggtgattgggtctccactgtagatgcatgcagtgttcactccaacctcagaggctttcttgcttctggggaaatggcaggtggttgaaaccctgcacatcctcccagggccagccccagaatgggcctttctctgcctgccctgcccttggggccacctctgcctccagcaggcttgagcttgccccaggccttgggcataattatttccccccaaaaaaggcacaaaaataggttttggatcaaagaagtgtttctcagtgaaatgaaaacagtctttttatagcctttagcttgtgagtttggaagtttggggggtcttatgtttgttttgcctcttctgtttcttggaggagagttgaggcttttcttaggtgcatacacagacccaggtgaacacgctgactgtgaacctgccctgtatccggagctgtgctgggcactgaggggatgcaacaaaattaggagaggatccttgctcccaacgtctacttctcctacctcaacaggggtccagggtgcagtgaactcagttcttggcccttgggtgaggattcatggatgaatgaaagctagacctgatggggaggcattatgactaaataggcccagcctccttcccttccagctctgtcctaggagcataggcgggaaatctgagtagagtctgactgcagtttttgcttatgatttgtaaaagccgtcatggggtcaataagaaaataggggtgatggagggggagaagcccaggactgggagaatcgcacatgccccaggggttttcaccaaggattttcaagacaaactggagtaagaattaaagccccagaggatttaattatcctggtttgcaaaagagcctcccatgccagtaccgcccagccttggaggccggaatgctcatggcccctgtggtctgcttgtccttcagcccatgcccagcagatacctctctgactggagacgggctcaaagctggattagaaaggggagaggcacttgtgactttgtttgactctgtgactcacttcctcgctcacaccttgtttgaactactggactttcaactggctttccttaggtcaggcaagcagacagctccccactgaagaggtctgtacagtgacaacccgggccggcagcaaggacacagatgcagccacagtaaggctccatcaggactgggtcagtgatggcaacaggatggccaaggatggctctagaacactctgtccatgcgtcactccccccagttttatttttagctttggcttcagggagtgacagccatcacaaatagccacattctgctctactctccaacataccagattctacactgttgttatttcatgagacgtgaatgttgcagagagtggggggattctggttgttaaggaacttacactggggagctttactcttccgtgtcaacaatgtgactacatgttctccagattagccacacatgcaaacatcagtgtccttctagctttagccgagaaagaaaccagtcccagggaatgaatggtggtctccccactcccggcagcactttaggcagcccataagctatgcgagaatgtgaacgctcaccttgctccgtcacggttctgacctaccacataaacaggaagaagccagtgaccggaacagctctaggaataacaagtcagaatagaagtgtcctttatattaccagaaaatatgggcttggcctaagtcgctgtctcctaacctgccggggtcattccccaccaaacaccccatactaaggagccatgagccacctggacattcaccttttctttgaccatctggagtctggggcaacttaaggaggcaccacacagtggtgcaggcacatttccaagcgtaggtgtccctggcttttgtggccaaagctagtgttatggtcaacaacaggccagggtctgtggggcactgaccttgaaagtggcaaaatggaggtttcacaggctgtgcgggagcaggacggcttgcttcatctaacaatctcagtttcctttaaaaaaagaaagaaaggaaaagatttcataagcaggtgtcagtggacagtttaagtacttaaccatttctctttcttcttatggatgtgaactgtgctgtggataaatcatttgtatttcttgaatgttctctatgactaacagttattaagtcggttgtgtatatgtgtaactaatgtaactgccttttaaaatttcattacaataaaaatgactttgctctgaacaa

SEQ ID NO: 17 - DNA, 298 bp, Homo sapiens
  source 1..298: mol_type = genomic DNA
ctgcggccagagtgaggctcctgctgtacagtgccacccatactgtgatgtcgatgcccatgtgaatgaggcccaccctcgccctgcttgccctgccgcaggccccccaccagaagccatctcccccgctgtgtgtccccaggcgcatcattgctccactctcccctgcagccgcggctgctcggccactgaccaggggccctggcagacattaggggatcagttgtatttattgtattttatctgtgtgtgctcaggaggtgagcctagtgggtccttaagtttggttgtgaataat

SEQ ID NO: 18 - DNA, 78 bp, Mus musculus
  source 1..78: mol_type = other DNA
ttgccctgctgtagaacccccagaaatctcccttattccccatcctccctggacatggcatcacctggctctaaccca

SEQ ID NO: 19 - DNA, 78 bp, Mus musculus; identical to SEQ ID NO: 18
  source 1..78: mol_type = other DNA

SEQ ID NO: 20 - DNA, 78 bp, Mus musculus; identical to SEQ ID NO: 18
  source 1..78: mol_type = other DNA

SEQ ID NO: 21 - DNA, 78 bp, Mus musculus; identical to SEQ ID NO: 18
  source 1..78: mol_type = other DNA

SEQ ID NO: 22 - DNA, 78 bp, Mus musculus; identical to SEQ ID NO: 18
  source 1..78: mol_type = other DNA

SEQ ID NO: 23 - DNA, 78 bp, Mus musculus; identical to SEQ ID NO: 18
  source 1..78: mol_type = other DNA

SEQ ID NO: 24 - DNA, 78 bp, Mus musculus; identical to SEQ ID NO: 18
  source 1..78: mol_type = other DNA

SEQ ID NO: 25 - DNA, 78 bp, Mus musculus; identical to SEQ ID NO: 18
  source 1..78: mol_type = other DNA

SEQ ID NO: 26 - DNA, 78 bp, Mus musculus; identical to SEQ ID NO: 18
  source 1..78: mol_type = other DNA

SEQ ID NO: 27 - DNA, 78 bp, Mus musculus
  source 1..78: mol_type = other DNA
ttgccctgctgcagaatccccagaaatctccctcattccccatcctccctggacatggcatcacctggctctaactca

SEQ ID NO: 28 - DNA, 78 bp, Mus musculus; identical to SEQ ID NO: 27
  source 1..78: mol_type = other DNA

SEQ ID NO: 29 - DNA, 78 bp, Mus musculus
  source 1..78: mol_type = other DNA
ttgccctgctgcagaatccccagaaatctccctnattccccatcctccctggacatggcatcacctggctctaacnca

SEQ ID NO: 30 - DNA, 78 bp, Mus musculus
  source 1..78: mol_type = other DNA
ttgccctgctgcagaatccccagaaatctccctcattccccatcctccctggacatggcatcacctggctctaaccca

SEQ ID NO: 31 - DNA, 78 bp, Mus musculus; identical to SEQ ID NO: 30
  source 1..78: mol_type = other DNA

SEQ ID NO: 32 - DNA, 78 bp, Mus musculus; identical to SEQ ID NO: 30
  source 1..78: mol_type = other DNA

SEQ ID NO: 33 - DNA, 78 bp, Mus musculus; identical to SEQ ID NO: 30
  source 1..78: mol_type = other DNA

SEQ ID NO: 34 - DNA, 78 bp, Mus musculus
  source 1..78: mol_type = other DNA
ttgccctgctgcagaatccccagaaatctccctcattccccatcctccctggacatggcatcacctggctctaacnca

SEQ ID NO: 35 - DNA, 78 bp, Mus musculus; identical to SEQ ID NO: 29
  source 1..78: mol_type = other DNA